(19) United States
(12) Patent Application Publication
(10) Pub. No.: US 2019/0048428 A1
(43) Pub. Date: Feb. 14, 2019

(54) PROCESSING BIOMASS

(71) Applicant: XYLECO, INC., Wakefield, MA (US)

(72) Inventors: Marshall Medoff, Wakefield, MA (US); Thomas Craig Masterman, Rockport, MA (US); John M. Cahill, Wakefield, MA (US)

(21) Appl. No.: 16/105,146

(22) Filed: Aug. 20, 2018

Related U.S. Application Data
(63) Continuation of application No. 14/897,947, filed on Dec. 11, 2015, now abandoned.
(60) Provisional application No. 62/026,742, filed on Jul. 21, 2014.
(60) Provisional application No. 62/027,489, filed on Jul. 22, 2014.

(51) Int. Cl. C13B 20/16; C13K 13/00; C12P 7/62; C12P 19/02

(52) U.S. Cl. CPC C13B 20/165; C13K 13/002; B01D 2317/025; B01D 2315/12; B01D 61/44; C12P 7/62; C13B 20/16; C12P 19/02; B01D 2311/2688

(57) ABSTRACT

Biomass feedstocks (e.g., plant biomass, animal biomass, and municipal waste biomass) are processed to produce useful products, such as fuels. For example, systems are described that can be useful for separating solids and high molecular weight species from liquids of bioprocessed biomass material slurries.

### CROSS REFERENCE TO RELATED APPLICATIONS

This application is a continuation of U.S. application Ser. No. 14/897,947, filed Dec. 11, 2015, which is a National Stage Entry of PCT Application No. PCT/US15/41306, filed Jul. 21, 2015, which claims the benefit of U.S. Provisional Application No. 62/027,489, filed Jul. 22, 2014 and U.S. Provisional Application No. 62/026,742, filed Jul. 21, 2014, the contents of each of which are hereby incorporated by reference.

### BACKGROUND OF THE INVENTION

Many potential lignocellulosic feedstocks are available today, including agricultural residues, woody biomass, municipal waste, oilseeds/cakes and seaweed, to name a few. At present, these materials are often under-utilized, being used, for example, as animal feed, biocompost materials, burned in a co-generation facility or even landfilled.

Lignocellulosic biomass includes crystalline cellulose fibrils embedded in a hemicellulose matrix, surrounded by lignin. This produces a compact matrix that is difficult to access by enzymes and other chemical, biochemical and/or biological processes. Cellulosic biomass materials (e.g., biomass material from which the lignin has been removed) are more accessible to enzymes and other conversion processes, but even so, naturally-occurring cellulosic materials often have low yields (relative to theoretical yields) when contacted with hydrolyzing enzymes. Lignocellulosic biomass is even more recalcitrant to enzyme attack. Furthermore, each type of lignocellulosic biomass has its own specific composition of cellulose, hemicellulose and lignin.

### SUMMARY

In general, processes for the filtering of materials, e.g., biomass materials, are disclosed herein. Processes are disclosed herein for saccharifying or liquifying a biomass material, e.g., cellulosic, lignocellulosic and/or starchy feedstocks, by converting biomass material to low molecular weight sugars. For example, processes are disclosed for saccharifying the feedstock, for example using an enzyme, e.g., one or more cellulase and/or amylase. The invention also relates to converting a feedstock to a product, e.g., by bioprocessing, such as fermentation or other processing, such as distillation. The processes include utilizing filtration to remove suspended solids, colored bodies, cells (e.g., yeast, bacteria) and/or viruses from biomass-derived liquids.

Provided herein is a purification method that includes producing a first permeate from a bioprocessed feedstock by retain material from the bioprocessed feedstock with a molecular weight above about a first molecular weight from the bioprocessed feedstock utilizing a first membrane filter. The method also includes producing a second permeate from the first permeate by retaining from the first permeate material with a molecular weight above about a second molecular weight from the first permeate utilizing a second membrane filter. The bioprocessed feedstock can be produced by saccharification of a biomass material and the saccharification can be done by contacting the biomass material with an enzyme or organism. Optionally the biomass material is a cellulosic or lignocellulosic material. Optionally the bioprocessed feedstock is in the form of a slurry that includes less than about 1% solids (e.g., less than about 0.5%, less than about 0.2%, less than about 0.1%). The solids can have a median particle size of less than about 10 μm (e.g., less than about 5 μm, less than about 1 μm). The bioprocessed feedstock can be filtered to remove the solids prior to utilizing the first membrane filter. Optionally, the bioprocessed feedstock includes at least about 1% solids (e.g. at least about 3% solids, at least about 9% solids) before being filtered. The bioprocessed feedstock can be filtered by a method selected from decanter centrifuging, disc centrifuging, stack filtration, plate filtration, micro filtration, column filtration, Vibratory Enhanced Separation Processes and combinations of these (e.g., two centrifuges utilized in series). Optionally, the bioprocessed feedstock is a fermentation product. Optionally, the bioprocessed feedstock is a distillation residue including at least one sugar. Some options include bioprocessed feedstock that comprises xylose (e.g., between about 0.1 and about 50% xylose, e.g., between about 0.5 and about 30% xylose, between about 1% and about 20% xylose, between about 1% and about 10%). In some options, the bioprocessed feedstock has had at least one volatile compound removed therefrom under vacuum prior to it being filtered by the first membrane filter. For example, the volatile compound can be an ester (e.g., a butyrate ester, a lactate ester) or an alcohol (e.g., ethanol, butanol). The concentration of alcohol in the bioprocessed feedstock can be less than about 5% (e.g., less than about 1%, less than about 0.5%, less than about 0.1%). Optionally, the first molecular weight is higher than the second molecular weight. Optionally, the first molecular weight can be at least about 100 kDa (e.g., at least about 150 kDa, at least about 200 kDa). In some options the first membrane filter retains particles larger than about 0.05 μm (e.g., larger than about 0.06 μm, larger than about 0.07 μm, larger than about 0.08 μm, larger than about 0.09 μm, larger than about 0.1 μm) from the bioprocessed feedstock. Optionally, the second molecular weight is at least about 2 kDa (e.g., between about 2 kDa and about 100 kDa, between about 2 kDa and about 50 kDa, between about 4 kDa and about 20 kDa). Optionally the first permeate has a lower turbidity (e.g., less than about 5 Nephelometric turbidity units, less than about 1 Nephelometric turbidity units) than the liquefied biomass (e.g., having at least about 5 NTU, having at least about 10 NTU, having at least about 50 NTU). Optionally the second permeate has less color (e.g., less than about 200 units by the Platinum-Cobalt ASTM Test Method D1209, less than about 100 units, less than about 50 units, less than about 40 units, less than about 30 units, less than about 20 units, less than about 10 units, less than about 5 units and even less than about 1 units) than the first permeate. Also provided herein are methods wherein the first and second membrane filters are configured as cross flow filters. For example, methods wherein the first and/or second membrane filters are spiral wound filters, tube filters or hollow fiber filters. For example, methods wherein the first and/or second membrane filters are configured as tube filters with a diameter between about ¼ and about 1 inch (e.g., about ½ inch). Optionally the inlet pressure at the first and/or the second membrane filter is between about 90 and about 500 PSIG (e.g., between about 100 and about 250 PSIG) and the outlet pressure at the first and/or second membrane filter is between about 20 and about 430 PSIG (e.g., between about 20 and 150 PSIG). Optionally the bioprocessed feedstock flows through the tube at a flow rate between about 1 and about 20 GPM (e.g., between about 2 and about 10 GPM, between about 4 to about 6 GPM). Optionally the first and/or second membrane filters are configured as modules including two or more bundles of tube filters (e.g., 7 or more, 19 or more, 37 or more tubes per module). For example wherein more than one module (e.g., 2, 3, 4, 5, 6 or more) is utilized to process the liquefied biomass material and/or the first permeate. Also provide herein are methods wherein the bioprocessed feedstock and the first permeate are at a temperature between about 30 and about 70 deg C. (e.g., between about 40 and about 65 deg. C., between about 40 and about 50 deg C.) when filtered through the first and second membrane filters. Optionally provided herein are methods further comprising concentrating the first permeate (e.g., utilizing an evaporator such as a triple effect evaporator, utilizing nanofiltration, utilizing reverse osmosis). Optionally provided herein are methods further comprising concentrating the second permeate (e.g., utilizing an evaporator such as a triple effect evaporator, utilizing nanofiltration, utilizing reverse osmosis). Optionally the second permeate is processed utilizing a system selected from the group consisting of an evaporator, electrodialysis, electrodialysis reversal, simulated moving bead chromatography, and combinations thereof.

Membrane separation technology is a useful alternative (or complementary method) to industrial separation methods such as distillation, centrifugation and extraction, since this technology potentially offers the advantages of highly selective separation, separation without any auxiliary materials, ambient or low temperature operation, no phase changes, continuous and automatic operation and economical operation. In addition, membrane separation units can be small and compact with modular construction and are simple and economical to integrate in existing production processes. Capital running cost is also relatively low. Thus, membrane separation processes can be useful for difficult to separate solutions that are biomass derived, for example, solutions that contain colloids fine particles, particles with density close to that of liquid phase, cells, proteins, polysaccharides, fermentation products, sugars and/or lignin.

Other features and advantages of the invention will be apparent from the following detailed description, and from the claims.

### DESCRIPTION OF THE DRAWINGS

The foregoing will be apparent from the following more particular description of example embodiments of the invention, as illustrated in the accompanying drawings. The drawings are not necessarily to scale, emphasis instead being placed upon illustrating embodiments of the present invention.

FIG. 1 shows a possible sequence for purification of a bioprocessed feedstock.

FIG. 2 shows schematically an embodiment of the inventive methods and flow of materials.

FIG. 3 shows schematically an embodiment of a tandem or cascading filtration system.

FIG. 4 is a plot of the particle size distribution of a fermented material.

FIG. 5 is a plot of the particle size distribution of a fermented and centrifuged material.

FIG. 6 is a plot of a particle size distribution of a fermented, centrifuged, heated and subsequently centrifuged material.

### DETAILED DESCRIPTION

Using the equipment, methods and systems described herein, cellulosic and lignocellulosic feedstock materials, for example that can be sourced from biomass (e.g., plant biomass, animal biomass, paper, and municipal waste biomass), can be turned into useful products and intermediates such as sugars and other products (e.g., fermentation products). Equipment, methods and systems to filter slurries utilizing membrane filtration are disclosed herein. For example, cross flow membrane filtration techniques are described, such as micro and ultra-filtration for upgrading biomass process streams.

Processes for manufacturing sugar solutions and products derived therefrom include, for example, optionally mechanically treating a cellulosic and/or lignocellulosic feedstock. Before and/or after this treatment, the feedstock can be treated with another physical treatment, for example irradiation, to reduce, or further reduce its recalcitrance. A sugar solution is formed by saccharifying the feedstock by, for example, the addition of one or more enzymes. A product can be derived from the sugar solution, for example, by selective fermentation of one or more of the sugars to an alcohol. Further processing can include purifying the solution, for example by centrifugation, rotary drum filtration, Vibratory Shear Enhanced Processes, distillation, ultrafiltration, electrodialysis, and/or simulated moving bed chromatography. For example, such further processing techniques are described in Attorney Docket No 00179-P1US filed at the same time as this application, PCT/US2014/21638 filed Mar. 7, 2014, PCT/US2014/21815 filed Mach 7, 2014 and PCT/US2014/21584 filed Mar. 7, 2014, the entire disclosures of which are incorporated herein by reference. If desired, the steps of measuring lignin content and setting or adjusting process parameters, (e.g., adjusting the pressure applied during filtration steps through membranes or the selection of membrane pore sizes/molecular weight cutoff) based on this measurement can be performed at various stages of the process. Some disclosure regarding adjusting process parameters is described in U.S. Pat. No. 8,415,122 issued Apr. 9, 2013, the complete disclosure of which is incorporated herein by reference.

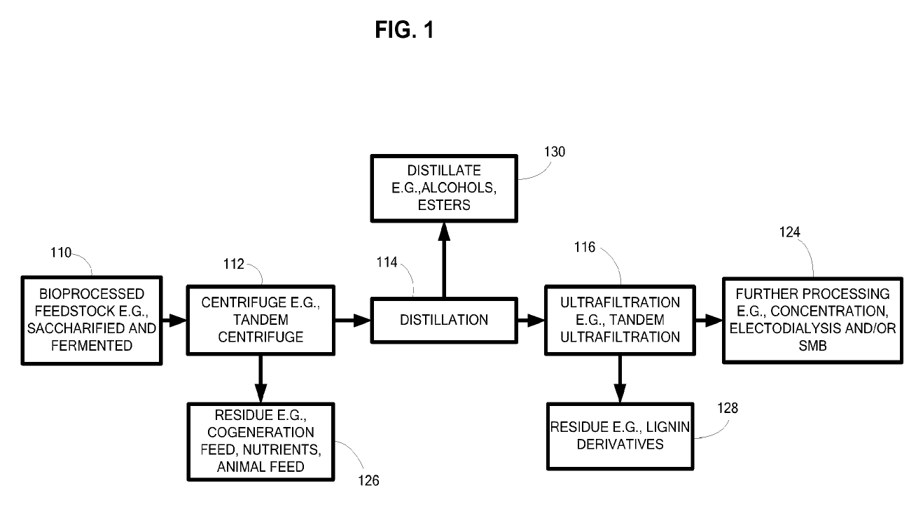

FIG. 1 shows a possible sequence for purification of a bioprocessed feedstock. A bioprocessed feedstock 110, for example a saccharified and fermented lignocellulosic or cellulosic material, is filtered by utilizing one or more centrifuges and/or Vibratory Shear Enhanced Processes (e.g., using a micron membrane filter) 112. Residues 126 can also be further processed, e.g., used as a feedstock for cogeneration of energy, as a nutrient (e.g., for a fermentation step), as an animal feed, as a fertilizer, and/or as an absorbent material. For example, the residues can be used in co-generation of energy as described in PCT/US2014/21634 filed Mar. 7, 2014, the entire disclosure of which is incorporated by reference herein. The filtrate material can be distilled 114 producing a distillate 130 such as purified alcohols or esters. The residue from the distillate can be filtered by ultrafiltration 116 using one ultrafiltration step or multiple ultrafiltration steps in series (e.g., 2, 3, 4, 5 or even more than 6). Residues from the ultrafiltration can be rich in molecular species such as lignin derivatives and can be used as a chemical feedstock or can be burned for energy (e.g., cogeneration). After the ultrafiltration, the solution (e.g., permeate) can be subjected to further processes 124. For example, further processing can include concentration, electrodialysis or electrodialysis reversal to remove ionic species, and/or a chromatographic method such as simulated moving bed chromatography to purify products such as sugars (e.g., xylose) or acids (e.g., lactic acid). These kinds of purifications are beneficial, and can be necessary because of the complexity of bioprocessed feedstocks that are biomass derived. Tandem ultrafiltration can be more efficient, faster and can reduce membrane fouling.

Since biomass is a complex feedstock, the composition and properties of the solids and the fluids derived therefrom can be complex and can vary greatly. For example, lignocellulosic materials include different combinations of cellulose, hemicellulose and lignin. Cellulose is a linear polymer of glucose. Hemicellulose is any of several heteropolymers, such as xylan, glucuronoxylan, arabinoxylans and xyloglucan. The primary sugar monomer present (e.g., present in the largest concentration) in hemicellulose is xylose, although other monomers such as mannose, galactose, rhamnose, arabinose and glucose are present. Although all lignins show variation in their composition, they have been described as an amorphous dendritic network polymer of phenyl propene units. The amounts of cellulose, hemicellulose and lignin in a specific biomass material depend on the source of the biomass material. For example wood-derived biomass can be about 38-49% cellulose, 7-26% hemicellulose and 23-34% lignin depending on the type. Grasses typically are 33-38% cellulose, 24-32% hemicellulose and 17-22% lignin. Clearly lignocellulosic biomass constitutes a large class of substrates.

The bioprocess feedstock 110 can be a suspension, such as a slurry, for example, a suspension of biomass particulates in a fluid (e.g., an aqueous solution). At least in part, the particulates are produced by mechanical treatments, for example mechanical treatments as described herein, e.g., that chop, grind, shear and/or comminute a biomass material such as a cellulosic and/or lignocellulosic material. These particulates of the slurry can have a wide range of properties. For example, the particulates can have a wide range of morphologies, for example, spheroid, ellipsoid, fibers, flakes, planar, smooth particles, rough particles, angular particles, cylindrical particles, fibrils, cellular (e.g., cells of any shape and size), conglomerates (e.g., a mass of dissimilar particles such as in size and/or shape), aggregates (e.g., a mass of similar particles such as in size and/or shape). The particulates also can have differing densities, for example having densities of between about 0.01 g/cc and greater than 5 g/cc (e.g., between about 0.1 and about 2 g/cc, between about 0.2 and about 1 g/cc). The particulates can have different or similar porosities, for example, in ranges between about 5% and about 90% (e.g., between about 5% and about 50%, between about 10% and about 40%). In addition to mechanical treatments, the properties of the particles and solutions in the feedstock slurry are determined by other processing steps such as, irradiation, saccharification and fermentation. A wide variety of size, shapes and kinds of particles and macromolecules present in a feed can make filtration difficult.

As noted, bioprocessing 110 can include saccharification of a recalcitrance reduced material. For example, recalcitrance reducing methods such as steam explosion, pyrolysis, oxidation, irradiation, sonication and combinations thereof can be utilized. Recalcitrance can also be reduced utilizing heat, such as applied by Jet cookers or other methods described in U.S. provisional Application Ser. No. 62/014,718 filed Jun. 20, 2014, the entire disclosure of which is incorporated by reference herein. Treatments, such as irradiation, can change the molecular weight of polymeric components by both chain scission and/or by cross-linking, depending on the treatment levels. Generally, above about 10 Mrad the treatments can reduce the molecular weights of cellulosic materials and also reduce the recalcitrance, e.g., make the material easier to saccharify. It is also possible that the irradiation reduces or increases the molecular weight of lignin components in the biomass. In addition to facilitating the saccharification, these treatments can modify the bioprocessed materials, for example by the molecular weight changes.

Saccharification can include suspending a biomass (e.g., a recalcitrance reduced biomass material) in water and treating the suspended biomass with heating (e.g., between about 80 and about 200 deg C., between about 100 and about 190 deg C., between about 120 and about 160 deg C.) and/or acids (e.g., mineral acids such as sulfuric acid). Other adjustments of pH with either acids or bases can further be used, adding to the ionic strength of the liquids. Optionally, or additionally, the saccharification can be accomplished by treatment with enzymes. For example, enzymes and biomass-destroying organisms that break down biomass, such as the cellulose, hemicellulose and/or the lignin portions of the biomass as described above, contain or manufacture various cellulolytic enzymes (cellulases), ligninases, xylanases, hemicellulases or various small molecule biomass-destroying metabolites. A cellulosic substrate is initially hydrolyzed by endoglucanases at random locations producing oligomeric intermediates. These intermediates are then substrates for exo-splitting glucanases such as cellobiohydrolase to produce cellobiose from the ends of the cellulose polymer. Cellobiose is a water-soluble 1,4-linked dimer of glucose. Finally cellobiase cleaves cellobiose to yield glucose. In the case of hemicellulose, a xylanase (e.g., hemicellulase) acts on this biopolymer and releases xylose as one of the possible products. Therefore, after saccharification the solution will have a high concentration of glucose and xylose and a concomitant decrease in cellulose and hemicellulose. For example if the slurry of saccharified biomass includes at least two monosaccharides (e.g., glucose and xylose) dissolved in the liquids, the monosaccharides concentration can include at least 50 wt. % of total carbohydrates available in the reduced recalcitrance cellulosic or lignocellulosic material, e.g., 60 wt. %, 70 wt. %, 80 wt. %, 90 wt. %, or even substantially 100 wt. %. Optionally, the glucose concentration can include least 10 wt % of the monosaccharides present in the saccharified material, e.g., at least 20 wt. %, 30 wt. %, 40 wt. %, 50 wt. %, 60 wt. %, 70 wt. %, 80 wt. %, 90 wt. % or substantially 100 wt. %. The remaining material in the slurry can include lignin and lignin derivatives that are dissolved or undissolved as well as dissolved and undissolved polysaccharides. For example, if the total amount of carbohydrates available in a saccharified material is 40 wt. % in a slurry of saccharified biomass, at least 50% of this material can be monosaccharides (e.g., which equates to a 20 wt. % monosaccharide in the saccharified biomass slurry) and of these monosaccharides, at least 10 wt. % can be glucose (e.g., at least 2 wt. %).

Bioprocessing 110 can also include fermentation, for example, fermentation after saccharification. For example, bioprocessing can include the fermentation of the sugars by the addition of an organism such as a yeast or bacteria to produce alcohols and acids (e.g., ethanol, butanol, acetic acid, lactic acid and/or butryric acid). Fermentation can also produce esters directly or the products chemically converted to esters. Fermentation can be a selective fermentation, e.g., fermenting only glucose or only xylose, or non-selective fermenting two or more sugars simultaneously or sequentially. The fermentation further changes the composition of the slurry, for example, by adding cellular debris from the fermentative organisms and fermentation by-products.

Therefore, the bioprocessed feedstocks that are derived from saccharification and fermentation of biomass can include various materials, for example suspended or dissolved compounds and/or materials. For example, solutions can include sugars, enzymes (e.g., parts of enzymes, active enzymes, denatured enzymes), amino acids, nutrients, live cells, dead cells, cellular debris (e.g., lysed cells, yeast extract), acids, bases, salts (e.g., halides, sulfates, and phosphates, alkali, alkali earth, transition metal salts), partial hydrolysis products (e.g., cellulose and hemicellulose fragments), lignin, lignin residues, inorganic solids (e.g., siliceous materials, clays, carbon black, metals), remnants of saccharified and/or fermented biomass, and combinations thereof. In addition, the sugar/fermented solutions can be colored due to colored impurities (e.g., colored bodies) such as aromatic chromophores. For example, some metal ions, polyphenols, and lignin-derived products produced or released during the processing of a lignocellulosic biomass can be highly colored. The color can be measured by a variety of methods, such as, the Platinum-Cobalt ASTM Test Method D1209. The methods described herein can, for example, reduce the color of the solutions to less than about 200 units by the Platinum-Cobalt test method (e.g., to less than about 100 units, less than about 50 units, less than about 40 units, less than about 30 units, less than about 20 units, less than about 10 units, less than about 5 units and even less than about 1 units).

The bioprocessed feedstock (e.g., slurry) can contain between about 1% and 20% total suspended solids (TSS) (e.g., between about 2% and about 10% solids, between about 3% and 9% solids). The TSS may be decreased, if desired, by centrifugation. After utilizing, for example the one or more centrifuges or Vibratory Shear Enhanced Processing (VSEP) 112 discussed above, the TSS is decreased to between about 0 and about 3% solids (e.g., between about 0 and 2%, between about 0.1 and about 1%). Preferably, the solids are less than about 1%. In addition to reducing the amount of solids, a filtering step (e.g., centrifuge and/or VSEP) can remove particles and modify the particle size distribution of the slurries. For example the first filtering step can remove most of the coarse particles, e.g., larger than 100 um (e.g., larger than about 50 um, larger than about 40 um, larger than about 30 um, larger than about 20 um). Therefore the median particle size after a first centrifuging step can be less than about 100 um (less than about 50 um, less than about 10 um or even less than about 5 um). A second centrifuge can remove smaller particles, e.g., between 100 um and 1 um. Therefore the median particle size after utilizing the second centrifuge can be between about 50 μm and 1 μm (e.g., between 10 and 1 μm, between about 5 μm and 1 μm). It is understood that some processes can be included that increase the particle size, modify the particle size distribution and/or increase the solids between one or more filtering steps or before the ultra-filtration step 116. Such processes may include, for example, denaturing of proteins, and/or addition of a precipitation or flocculating agent. For example, heating during distillation, if implemented between or after filtering steps, can denature proteins as well as concentrate solids causing coagulation, flocculation and/or agglomeration.

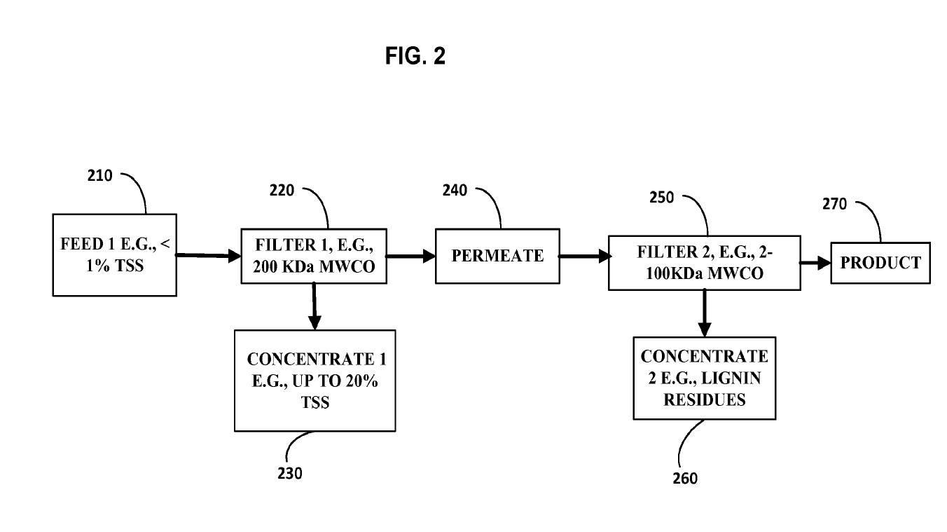

FIG. 2 shows schematically an embodiment of a method that can be utilized and the flow of materials. In this embodiment, a feed 210 that is derived from a filtering (e.g., centrifuge and/or VSEP step) 112 can include a low amount of total suspended solids (TSS), for example less than about 3% solids (e.g., with one centrifuge step) or even lower (e.g., with one or more centrifuge steps), such as less than about 1% (e.g., less than about 0.5%, less than about 0.2%, less than about 0.1%). The percent solids refer to a wt % of dry solids (e.g., dry solids weight/slurry weight×100% as described in the examples).

A first membrane filtration 220 can be targeted to retain material in a concentrate (or exclude material from a permeate) with a molecular weight above about 100 kDa, (e.g., above about 150 kDa, about 200 kDa.) In an alternative description, the first membrane filtration 220 can be targeted to retain particulates in a concentrate (or exclude particulates from a permeate) with a particle size greater than about 0.05 μm (e.g., greater than about 0.06 μm, greater than about 0.07 μm, greater than about 0.08 μm, greater than about 0.09 μm, greater than about 0.1 μm). Since microfiltration can be classified as the removal of materials between about 0.08 and about 4 μm from a feed stream without strictly defined boundaries, the first filtration can be done using a microfiltration configuration in some embodiments. However, it is preferred that an ultrafiltration configuration is utilized, where ultrafiltration generally relates to removal of particle sizes below about 0.3 μm (molecular weight about 300 kDa) and above about 0.005 μm and about 50 kDa from a feed steam.

The first membrane filtration 220 produces a permeate 240 and a first concentrate 230. The first concentrate will have a higher TSS than the feed, e.g., up to about 20% TSS (e.g., up to about 10 wt. % solids, up to about 5 wt. % solids). The first concentrate will also have most of the molecular species (e.g., greater than about 95 wt. %, greater than about 99 wt. %, greater than about 99.9 wt. %) with a molecular weight above the molecular weight cutoff of the first membrane as described above. The first permeate 240 will have a very low TSS (e.g., less than about 0.05 wt. %, less than about 0.01 wt. %, about 0 wt. %) and substantially no molecular species above the molecular weight cut off of the first membrane (e.g., less than 5 wt. %, less than about 1 wt. %, less than about 0.1 wt. %). Therefore, the first permeate has a greatly reduced turbidity as compared to the feed. For example, the feed can have a turbidity of at least about 50 Nephelometric turbidity units (NTU) (e.g., at least about 10 NTU, at least 5 NTU) and the first permeate can have a turbidity of less than about 5 NTU (e.g., less than about 1 NTU). When the filtration is complete, the first concentrate stream will typically have a volume less than about 20% of the feed volume (e.g., less than about 10%, less than about 5% or even less than about 1%). When the filtration is complete, the permeate 240 typically will have a volume that is more than about 80% of the feed volume (e.g., more than about 90%, more than about 95%). Some losses of the total volume (e.g., due to spilling, flushing of the system or components, priming) can occur although these are generally less than 5% (e.g., less than about 1%). The first filtration is expected to remove any yeast or bacterial cells that may be present in the feed, so that the permeate (e.g., filtered material) can be sterile and the concentrate (e.g., non-filtered material) can contain yeast or bacterial cells present from the feed stream.

The first permeate can be filtered by a second filter 250 and this filtration step creates a second concentrate 260 and second permeate or product 270. The second concentrate stream, when the filtration is completed, will typically have a volume of less than about 20% of the feed volume e.g., less than about 10%, less than about 5% or even less than about 1%). The second permeate or product volume, when the filtration is completed, typically will have more than about 80% of the feed volume (e.g., more than about 90%, more than about 95%). As in the first filtration, some losses of the total volume can occur although these are generally less than 5% (e.g., less than about 1%). The second filtration is targeted to retain in a concentrate (or exclude from a permeate) material larger than about 5 kDa (e.g., at least 10 kDa, at least 20 kDa, such as between 10 and 100 kDa). Although there are preferably almost no solids in the first permeate (e.g., no particulates above about 0.1 μm could pass through the first membrane), the second filter can retain in a concentrate (or exclude from a permeate) any remaining solids or particulates above the size of 50 nm (e.g., above about 10 nm, above about 5 nm) depending on the membrane size selected. The second filtration can therefore remove many or even all viruses as well as oligomers and polymers and large molecules or inorganic clusters. The second filtration can also decolorize the first permeate since, as discussed above, color bodies can be due to large molecules such as aromatic chromophors derived, for example, from lignin. The second filtration does not remove most ionic species and small molecules such as monomeric and dimeric sugars.

Additional filtration steps can be included, for example, to filter the product 270. For example, nanofiltration and reverse osmosis can be utilized. For example, nanofiltration can be used to separate sugars from ionic species and concentrate the sugars. Therefore, a nanofiltration step can produce a sugar solution of high purity (e.g., at least about 90 mol. % pure excluding water, at least about 95 mol %, at least about 99 mol %, at least about 99.9 mol %) that is of high concentration (e.g., with a sugar concentration greater than about 10 wt. %, greater than about 15 wt. %, greater than about 20 wt. %, greater than about 25 wt. %, greater than about 30 wt. %, greater than about 35 wt. %, greater than about 40 wt. %, greater than about 45 wt. %, greater than about 50 wt. %) and a permeate with metal ions. Reverse osmosis can produce a pure water permeate (e,g., at least 90 mol % water, at least 95 mol %, at least 99 mol %, at least 99.9 mol %), and a concentrate containing sugars (e.g., at least about 90 mol. % sugar excluding water and ions, at least about 95 mol %, at least about 99 mol %, at least about 99.9 mol %) and ions.

As previously described, membrane separation technologies can be useful for separation (e.g., upgrading) of biomass derived materials such as feed 210. Preferably, the filtration is a cross flow filtration such as the microfiltration, ultrafiltration, nano-filtration or reverse osmosis methods described above. In conventional filtration (e.g., dead end filtration) the feed flow is perpendicular to the membrane surface, which causes a buildup of debris that eventually reduces fluid permeation due to prohibitive pressure buildup that can lead to membrane rupture. In cross flow filtration, flow is tangential to the membrane surface, resulting in a continuous scouring action that almost eliminates formation of a membrane fouling layer from feed-stream debris and macro molecules.

Several different configurations can be utilized for cross flow filtration. For example, hollow fiber membranes can include a bundle of many (e.g., more than 100 fibers) in an enclosed tube. Hollow fibers range from 0.019 to 0.118 inch (0.5 to 3 mm) ID and make it possible for a large surface area to be housed in a small volume. Due to the size of the hollow fibers, generally these filters are suitable for materials with very low TSS and only small particle sizes, for example, for water purification in drinking water applications. Spiral wound membranes are also a compact membrane format that can operate at high pressures and are often used for solutions with low amounts of suspended solids. In this configuration the filtering membrane is wound around a hollow inner tube with spacers for passage of the solution. The permeate passes through the membrane and to the inner core due to the spirally wound configuration. A configuration known as a “tubular” includes one or more tubular membranes inside a tube housing (e.g., stainless steel). The tubular membranes can have internal diameters ranging from about ¼ to about 1 inch. These can be made from polymeric or ceramic materials. Often, to improve throughput, several tubular membranes are bundled inside the tubular housing, for example 7, 19, 37 or more arranged in a honeycomb structure as seen down the long axis or flow direction. Membranes can be made of polymeric materials, for example cellulose acetate (CA), polyvinylidene fluoride (PVDF), polyacrylonitrile (PAN), polypropylene (PP), polysulfone (PS), polyethersulfone (PES), or other polymers. The membranes can also be made of ceramic materials such as metal oxides of titanium, zirconium, aluminum and silica. In some configurations polymeric tubes are glued together into a bundle. Ceramic membranes are often a single monolith with multiple channels or tubes through the monolith.

In some embodiments the first and second filtration steps utilize tubular membrane ultrafiltration. In other embodiments at least the first filtration step utilizes a tubular membrane filtration device while subsequent filtration steps can use a spiral wound or microfiber membrane filtration configuration.

During operation, the inlet pressure, using ultrafiltration, can be between about 90 and about 500 PSIG (e.g., between about 100 to about 250 PSIG) and the outlet pressure is expected to drop to between about 20 and about 430 PSIG (e.g., between about 20 and about 150 PSIG). During operation, the difference between the inlet and outlet pressures can be, for example, between about 70 and about 120 PSIG). For example, the aforementioned pressures, can relate to a pass of 10 to 12 membrane modules in series, each module having tubular configuration of ½ inch diameter tubes in a 19 tube bundle at least twelve feet long.

The operating temperature can be between about 30 and about 70 deg C. (e.g., between about 40 and about 65 deg. C., between about 40 and about 50 deg C.). The operating temperature can be controlled, for example, using a heater or chiller (e.g., heat exchangers). Higher temperatures can increase the permeate flow rates. If the membranes become fouled and the permeate throughput decreases (e.g., less than about 1% of the flow into the filtration unit, e.g., less than about 0.5%, less than about 0.1%) the membranes can be cleaned by flowing a cleaning solution (e.g., a caustic solution) to flush the membranes. The cleaning solutions can also be heated and controlled similarly to the process fluids.

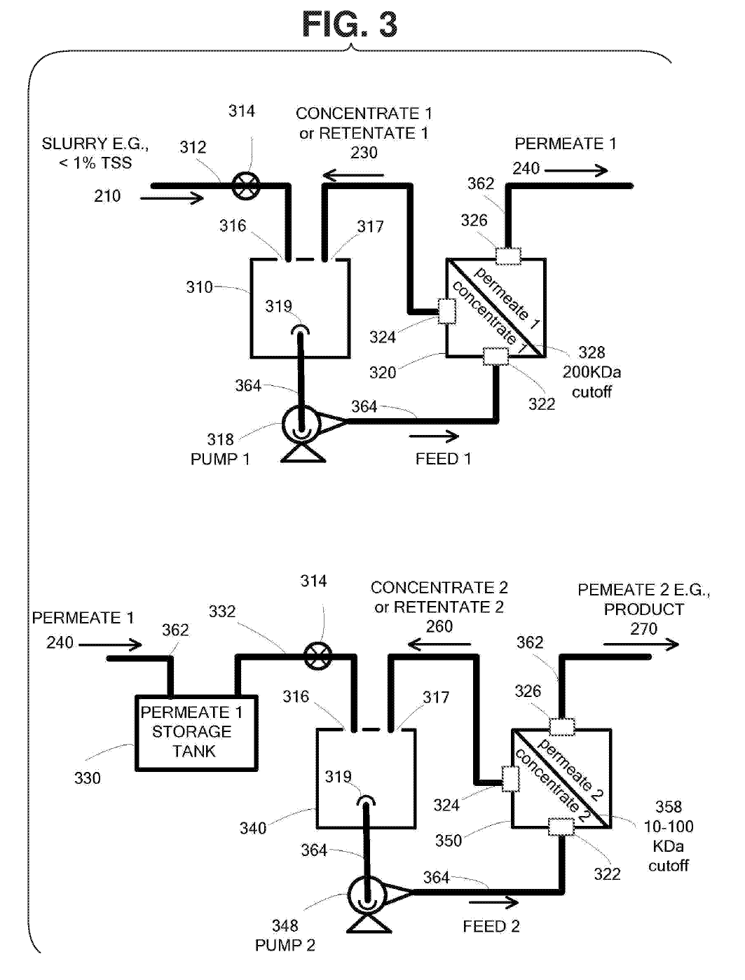

FIG. 3 shows schematically an embodiment of a tandem or cascading filtration system 300 and methods of its use. For example first feed tank 310 is charged with a feed 210 (e.g., containing less than 1% TSS). The feed tank can be filled, for example, through a tube or pipe 312 fit with a flow control valve 314 and fluidly connected to the tank through an inlet 316. When the first tank is charged to the desired level (e.g., at least 90% of the internal volume, at least 50% of the internal volume) the flow of slurry 210 can be shut off or reduced by the control valve. The first pump 318 can then be activated if it is not already on. The pump drives fluids from the first feed tank, through the first membrane filtration unit 320 and back to the feed tank through inlet 317. The pump 318 provides the pressure (e.g., inlet pressure) that forces the process liquids through the membrane tubes and some of the liquids pass through the membrane in the first membrane filter unit, forming the first permeate 240 that flows through a tube 362. The pump is fluidly connected through an outlet 319 to the first feed tank, and through tubes 364 to an inlet 322 of the first membrane filter unit 320. The first membrane filter unit is shown only schematically in FIG. 3, wherein the diagonal line 328 represents a membrane filter with a 200 kDa cutoff, separating a concentrate side and a permeate side. In practice, the configurations of the first membrane filter unit can include, for example a series of passes of ½ inch tubes bundled into membrane modules with u-bends connecting each module (e.g., the u-bends allow for a more compact 3-D arrangement). The length of the membrane modules (e.g., series of modules) that the solutions pass through is between about 120 to about 144 feet. For example, the first membrane filter unit can include about 10 to about 12 modules, each containing a bundle of 37 tubes that are each about 12 feet long. The membrane filter unit configurations should generally include an inlet 322 that is an inlet to all of the tubular membranes, an outlet 324 that is the outlet for the concentrate/retentate of all the tubular membranes, and an outlet 326 that is the outlet for the permeates of all the tubular membranes. The outlet 324 is connected to the retentate return tube 230 and to the inlet 317.

The first permeate 240 is fed to a storage tank 330 (e.g., overflow tank or buffer tank) through tubes 362. The storage tank is connected through tubes 362 to the first filtration unit, and through tube 332 to the second feed tank 340. Optionally, the first permeate 240 in the storage tank is pumped using a pump (not shown) through tube 332. Alternatively or additionally, the first permeate can be made to flow from the storage tank to the second feed tank by gravity. The second feed tank 340 can be configured similarly to the first feed tank, for example, with inlets 316 and 317, outlet 319, a second pump 348 and fluid connecting tubes 332 and 364. The second membrane filtration unit 350 can be configured similarly to the first membrane filtration unit. Preferably the second membrane 358 in the second membrane filtration unit 350 has a lower molecular weight and/or particle size cut off than the first membrane 328 in the first filtration unit 324, for example a membrane 358 can be selected to have a molecular weight cutoff between about 2 and 100 kDa (e.g., between 10 and 100 kDa). The permeate from the second filtration (e.g., second permeate or product) 270, can be sent to a storage tank (not shown) and/or to other processing through a tube.

During operation of each unit, the feed material is circulated at a high rate, for example so that the material flows through each tube of the tubular membrane at a rate of at least 1 GPM, e.g., a flow rate between about 1 and about 20 GPM (e.g., between about 2 and about 10 GPM, between about 4 to about 6 GPM). Only a fraction of this flow passes through the membrane, for example between about 1 and 10% of the flow becomes (or forms) permeate depending on the slurry compositions (e.g., composition properties such as the TSS and molecular species present) and the selected membrane (e.g., the molecular weight and/or particle size cut off). The inlet pressures to the membranes are carefully monitored and are targeted to be between 90 and about 500 PSIG. Outlet pressures are likewise monitored, with target ranges between about 20 and about 143 PSIG. The pressures can be adjusted by throttling an outlet valve or adjusting the pump speed (e.g., using a variable frequency drive, VFD).

The separating process of the membrane filters and recirculating flows cause the fluids in the feed tanks to increase in concentration of particulates or molecules that cannot pass through the selected membrane, causing the concentrate in the feed tank to have ever increasing levels of membrane-excluded particulates and/or molecular species. When the feed tank has been reduced to about 10% of the original volume, the filtration can be considered completed and the concentrate can be removed from the first feed tank 310, or second feed tank 340 and the tanks can be refilled with slurry 210 or permeate 240 respectively.

System 300 can be operated in a batch-wise fashion. For example, in the batch method first feed tank 310 is filled and processed through the first membrane filter unit when the desired amount of permeate has been collected to storage tank 330 and/or the feed tank volume in tank 310 is reduced to the desired target. Once the batch is processed, the first feed tank can be refilled. The second membrane filtration can be operated similarly, utilizing the material in the permeate storage tank to fill the second feed tank 340. The processing rates of the membrane filter units are optimally balanced to have minimum down time. For example, the feed tanks in some preferred embodiments of batch operation are filled at the same time and the both membrane filter units are run at the same time. To run this process it can be necessary to run the two membrane filter units differently (e.g., at different pressures) and/or use different configurations of filter membrane tubes (e.g., different tube lengths and/or different numbers of parallel tubes). The process can also be run with one first filtration step or system (e.g., such as using one filter unit 320) and then branching this single stream to two, three, four or more second filtration step or systems (e.g., such as using two, three, four or more second filter unit 350) operated in parallel. Optionally, the process can also be done using two, three, four or more first filtration steps in parallel and then combining these multiple streams into a single stream and using a second filtration step.

System 300 can also be run in a semi-continuous method. For example, the feed tanks can be supplemented with slurry 210 or permeate 240 respectively as the volumes are reduced. When the concentration of species in the concentrates 230 and/or 260 are at a targeted value, the concentrate is removed from the respective feed tank and replaced with fresh slurry or permeate respectively. The targeted value can be determined, for example, by analysis of the feed tank solutions (e.g., for turbidity, particle concentrations and/or chemical composition) and/or by monitoring the pressure changes at the filter membranes (e.g., when the inlet pressure reaches a set value, such as more than about 100 psi, more than about 120 psi, or more than about 150 psi, the concentrate can be determined no longer processable through the membrane).

The feed tanks and storage tank can be scaled for processing different volumes. For example, in some embodiments the system 300 is designed to process about 330 kgal per day (e.g., about 230 gal per minute). The feed tanks therefore can be designed to accommodate 330 kgal if run in a batch mode. In some embodiments the feed tanks can be divided into several tanks, for example, three 100 kgal or six 50 kgal tanks. Other configurations can be designed, for example, for processing small volumes such as between about 100 gal and 100 kgal per day (e.g., between 50 and 500 gal, between 500 gal and 1000 gal, between 1000 gal and 10000 gal). Other configurations can be designed to process more than 500 kgal/day e.g., more than 1000 kgal/day.

### EXPERIMENTAL

### Saccharification

A cylindrical tank with a diameter of 32 Inches, 64 Inches in height and fit with ASME dished heads (top and bottom) was used in the saccharification. The tank was also equipped with a hydrofoil-mixing blade 16″ wide. Heating was provided by flowing hot water through a half pipe jacket surrounding the tank.

The tank was charged with 200 kg water, 80 kg of biomass, and 18 kg of DUET™ Cellulase enzyme. Biomass was corncob that had been hammer milled and screened to a size of between 40 and 10 mesh. The biomass had also been irradiated with an electron beam to a total dosage of 35 Mrad. The pH of the mixture was adjusted and maintained automatically throughout the saccharification at 4.8 using Ca(OH)₂. This combination was heated to 53 deg. C., stirred at 180 rpm (1.8 Amp at 460V) for about 24 hours after which the saccharification was considered completed.

A portion of this material was screened through a 20-mesh screen and the solution stored in an 8 gal carboy at 4 deg. C.

Biomass Produced Ethanol and Xylose Stream

About 400 mL of the saccharified and screened material was decanted into a 1 L New Brunswick BioFlow 115 Bioreactor. The material was aerated and heated to 30 deg. C. prior to inoculation. Stirring was set at 50 rpm. The pH was measured at 5.2, which is acceptable for fermentation so it was not adjusted. Aeration was discontinued and the contents of the bioreactor were inoculated with 5 mg of THERMOSACC® Dry Yeast (Lallemand, Inc.). Fermentation was allowed to proceed for about 24 hours.

After fermentation the glucose concentration was below the detection limit, the ethanol concentration was about 25 g/L, and the xylose concentration was 30 g/L.

### Centrifuge Experiments

Corn cob was saccharified and fermented similarly to the above but at a larger scale (300 gal). In addition the corn cob was pre-treated (before enzyme hydrolysis) by heating at between 100 and 160 C. The percent solids and particle size data in Table 1 below was obtained from 3 process stream samples: A. after fermentation, B. after using a decanter centrifuge, and C. after taking the decanter centrifuged material, heating it to about 90 deg C., and utilizing a disk centrifuge to further process the material. It is expected that a second high speed decanter centrifuge can give a similar particle size distribution and decrease in the total suspended solids (TSS) as a disk centrifuge.

The Decanter centerfuge (US centrifuge) was operated at 2000 g of centrifugal force and processed material at between 25 and 100 gal/min.

The disk centrifuge was a Clara 80 Low Flow centrifuge (Alfalaval) fit with a 567723-06/-08 bowl. The centrifuge was run at between about 7000 and 8000 rpm processing about 0.5 to 1 gal/min.

Each sample was prepared as follows. A 50.0 mL sample was tared and then filtered using Corning filters (part 431117) to produce a filter cake. The cake was dried 3 times with DI water and then dried overnight (approximately 18 hrs) in a vacuum oven (Fisher Isotemp Model 281A) at 70 deg C. and 29 inches Hg. After drying, the dried cakes were weighed. The total suspended solids (TSS) was calculated by weight and volume and is recorded in Table 1

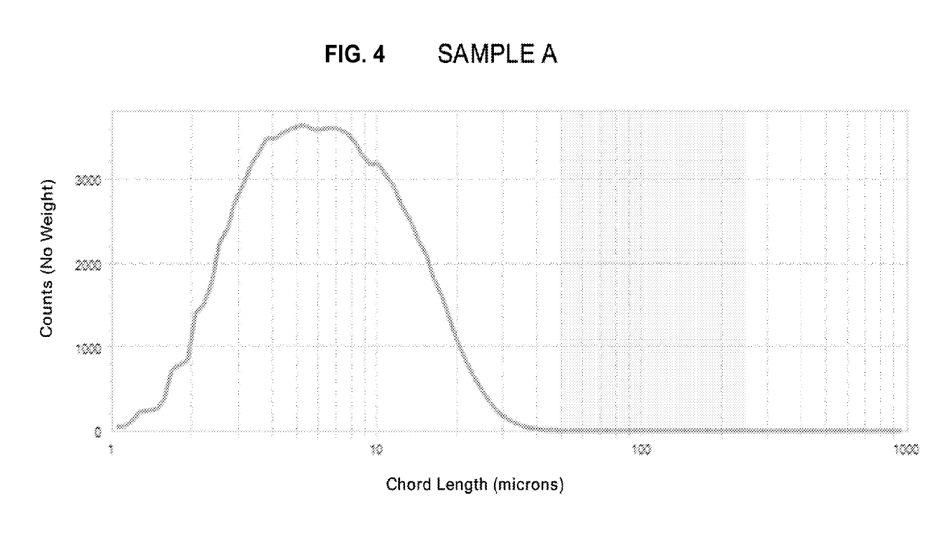

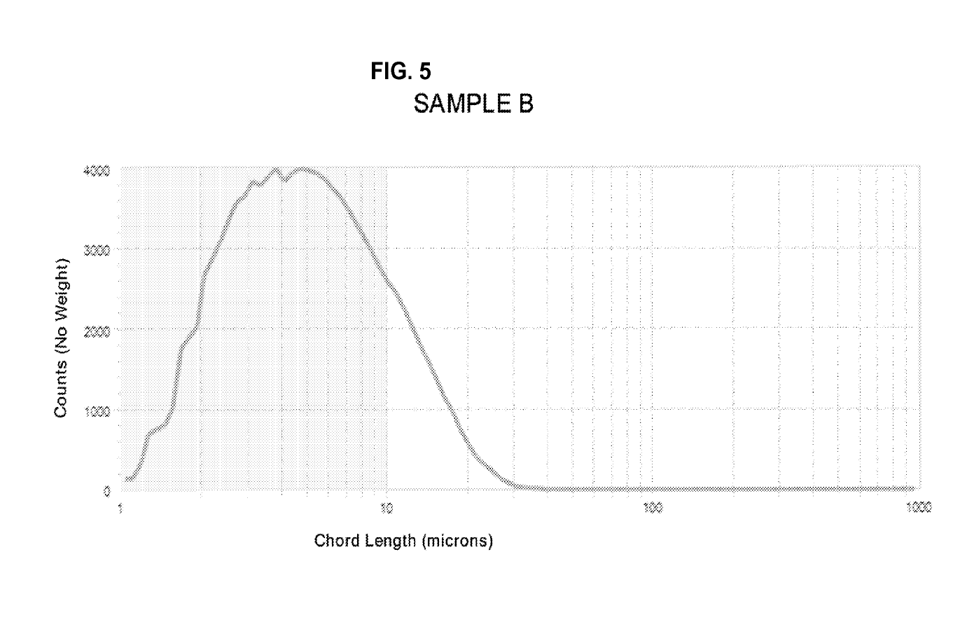

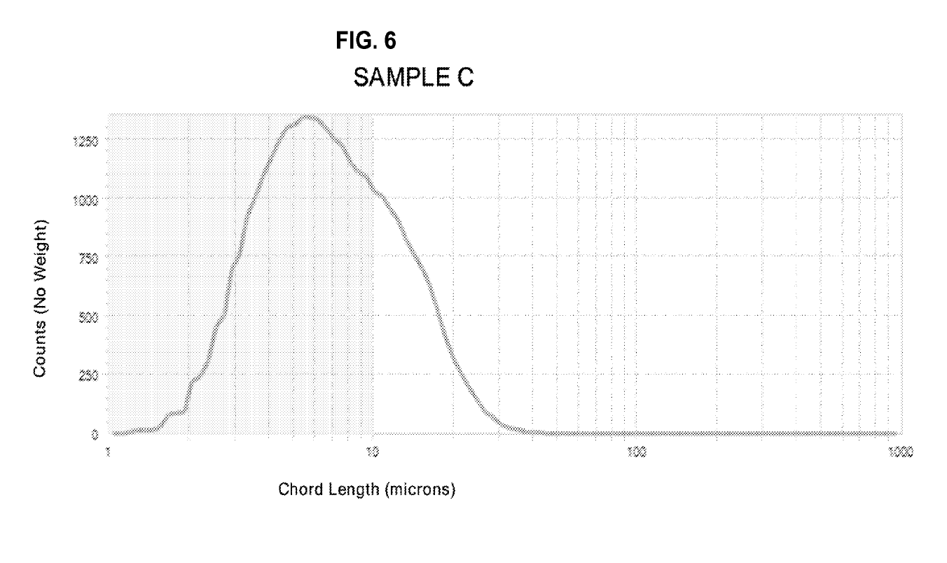

In addition to the TSS, samples were taken for particle size analysis using a Mettler Toledo Focused Beam Reflectance measurement Model Particle Trace E25. The median particle size is recorded in Table 1. The particle size distributions are plotted as FIG. 4 for sample A, FIG. 5 for sample B, and FIG. 6 for sample C.

As can be seen from Table 1, centrifuging once utilizing a decanter centrifuge resulted in about a 50% reduction in the solids level. A second centrifuging step can reduce the solids level further, e.g., from about 3% to about 0.2%.

TABLE 1

| Sample | Solids % wt/wt | % Solids wt/vol | Median particle Size (μm) |
|---|---|---|---|
| A | 6.1 | 6.4 | 6.12 |
| B | 3.0 | 3.2 | 4.8 |
| C | 0.21 | 0.22 | 6.53 |

### Ultrafiltration Experiments

Ultrafiltration can be utilized to purify the process stream from the decanter centrifuge provided as described above. Fifty gallons of the process stream from the decanter centrifuge, containing about 0.21% solids, can be processed in a pilot run tangential ultrafiltration system. Ultrafiltation membranes include single pass using a A37 tublar membrane module (PCI membranes, Hamilton, Ohio). The first membrane filter can be a tubular membrane having a 200 kDa (˜0.1 μm) cut off. The material is processed through this membrane at a feed rate of between about 5 and 6 GPM. After about 24 hours, filtration through this first module is complete, producing about 90 gal of permeate and 10 gal of concentrate. The 90 gal of permeate are processed through a single pass using A37 tubular membrane module with a 20 kDa cutoff at a rate of between 5 and 6 GPM. This processing produces about 80 gal of permeate product and 10 gal of concentrate.

### Radiation Treatment

The feedstock, such as a lignocellulosic or cellulosic material, can be treated with radiation to modify its structure to reduce its recalcitrance. Such treatment can, for example, reduce the average molecular weight of the feedstock, change the crystalline structure of the feedstock, and/or increase the surface area and/or porosity of the feedstock. Radiation can be by, for example electron beam, ion beam, 100 nm to 28 nm ultraviolet (UV) light, gamma or X-ray radiation. Radiation treatments and systems for treatments are discussed in U.S. Pat. No. 8,142,620 and U.S. patent application Ser. No. 12/417,731, the entire disclosures of which are incorporated herein by reference.

Each form of radiation ionizes the biomass via particular interactions, as determined by the energy of the radiation. Heavy charged particles primarily ionize matter via Coulomb scattering; furthermore, these interactions produce energetic electrons that may further ionize matter. Alpha particles are identical to the nucleus of a helium atom and are produced by the alpha decay of various radioactive nuclei, such as isotopes of bismuth, polonium, astatine, radon, francium, radium, several actinides, such as actinium, thorium, uranium, neptunium, curium, californium, americium, and plutonium. Electrons interact via Coulomb scattering and bremsstrahlung radiation produced by changes in the velocity of electrons.

When particles are utilized, they can be neutral (uncharged), positively charged or negatively charged. When charged, the charged particles can bear a single positive or negative charge, or multiple charges, e.g., one, two, three or even four or more charges. In instances in which chain scission is desired to change the molecular structure of the carbohydrate containing material, positively charged particles may be desirable, in part, due to their acidic nature. When particles are utilized, the particles can have the mass of a resting electron, or greater, e.g., 500, 1000, 1500, or 2000 or more times the mass of a resting electron. For example, the particles can have a mass of from about 1 atomic unit to about 150 atomic units, e.g., from about 1 atomic unit to about 50 atomic units, or from about 1 to about 25, e.g., 1, 2, 3, 4, 5, 10, 12 or 15 atomic units.

Gamma radiation has the advantage of a significant penetration depth into a variety of material in the sample.

In embodiments in which the irradiating is performed with electromagnetic radiation, the electromagnetic radiation can have, e.g., energy per photon (in electron volts) of greater than 10² eV, e.g., greater than 10³, 10⁴, 10⁵, 10⁶, or even greater than 10⁷ eV. In some embodiments, the electromagnetic radiation has energy per photon of between 10⁴ and 10⁷, e.g., between 10⁵ and 10⁶ eV. The electromagnetic radiation can have a frequency of, e.g., greater than 10¹⁶ Hz, greater than 10¹⁷ Hz, 10¹⁸, 10¹⁹, 10²⁰, or even greater than 10²¹ Hz. In some embodiments, the electromagnetic radiation has a frequency of between 10¹⁸ and 10²² Hz, e.g., between 10¹⁹ to 10²¹ Hz.

Electron bombardment may be performed using an electron beam device that has a nominal energy of less than 10 MeV, e.g., less than 7 MeV, less than 5 MeV, or less than 2 MeV, e.g., from about 0.5 to 1.5 MeV, from about 0.8 to 1.8 MeV, or from about 0.7 to 1 MeV. In some implementations the nominal energy is about 500 to 800 keV.

The electron beam may have a relatively high total beam power (the combined beam power of all accelerating heads, or, if multiple accelerators are used, of all accelerators and all heads), e.g., at least 25 kW, e.g., at least 30, 40, 50, 60, 65, 70, 80, 100, 125, or 150 kW. In some cases, the power is even as high as 500 kW, 750 kW, or even 1000 kW or more. In some cases the electron beam has a beam power of 1200 kW or more, e.g., 1400, 1600, 1800, or even 300 kW.

This high total beam power is usually achieved by utilizing multiple accelerating heads. For example, the electron beam device may include two, four, or more accelerating heads. The use of multiple heads, each of which has a relatively low beam power, prevents excessive temperature rise in the material, thereby preventing burning of the material, and also increases the uniformity of the dose through the thickness of the layer of material.

It is generally preferred that the bed of biomass material has a relatively uniform thickness. In some embodiments the thickness is less than about 1 inch (e.g., less than about 0.75 inches, less than about 0.5 inches, less than about 0.25 inches, less than about 0.1 inches, between about 0.1 and 1 inch, between about 0.2 and 0.3 inches).

It is desirable to treat the material as quickly as possible. In general, it is preferred that treatment be performed at a dose rate of greater than about 0.25 Mrad per second, e.g., greater than about 0.5, 0.75, 1, 1.5, 2, 5, 7, 10, 12, 15, or even greater than about 20 Mrad per second, e.g., about 0.25 to 2 Mrad per second. Higher dose rates allow a higher throughput for a target (e.g., the desired) dose. Higher dose rates generally require higher line speeds, to avoid thermal decomposition of the material. In one implementation, the accelerator is set for 3 MeV, 50 mA beam current, and the line speed is 24 feet/minute, for a sample thickness of about 20 mm (e.g., comminuted corn cob material with a bulk density of 0.5 g/cm³).

In some embodiments, electron bombardment is performed until the material receives a total dose of at least 0.1 Mrad, 0.25 Mrad, 1 Mrad, 5 Mrad, e.g., at least 10, 20, 30 or at least 40 Mrad. In some embodiments, the treatment is performed until the material receives a dose of from about 10 Mrad to about 50 Mrad, e.g., from about 20 Mrad to about 40 Mrad, or from about 25 Mrad to about 30 Mrad. In some implementations, a total dose of 25 to 35 Mrad is preferred, applied ideally over a couple of passes, e.g., at 5 Mrad/pass with each pass being applied for about one second. Cooling methods, systems and equipment can be used before, during, after and in between radiations, for example utilizing a cooling screw conveyor and/or a cooled vibratory conveyor.

Using multiple heads as discussed above, the material can be treated in multiple passes, for example, two passes at 10 to 20 Mrad/pass, e.g., 12 to 18 Mrad/pass, separated by a few seconds of cool-down, or three passes of 7 to 12 Mrad/pass, e.g., 5 to 20 Mrad/pass, 10 to 40 Mrad/pass, 9 to 11 Mrad/pass. As discussed herein, treating the material with several relatively low doses, rather than one high dose, tends to prevent overheating of the material and also increases dose uniformity through the thickness of the material. In some implementations, the material is stirred or otherwise mixed during or after each pass and then smoothed into a uniform layer again before the next pass, to further enhance treatment uniformity.

In some embodiments, electrons are accelerated to, for example, a speed of greater than 75 percent of the speed of light, e.g., greater than 85, 90, 95, or 99 percent of the speed of light.

In some embodiments, any processing described herein occurs on lignocellulosic material that remains dry as acquired or that has been dried, e.g., using heat and/or reduced pressure. For example, in some embodiments, the cellulosic and/or lignocellulosic material has less than about 25 wt. % retained water, measured at 25° C. and at fifty percent relative humidity (e.g., less than about 20 wt. %, less than about 15 wt. %, less than about 14 wt. %, less than about 13 wt. %, less than about 12 wt. %, less than about 10 wt. %, less than about 9 wt. %, less than about 8 wt. %, less than about 7 wt. %, less than about 6 wt. %, less than about 5 wt. %, less than about 4 wt. %, less than about 3 wt. %, less than about 2 wt. %, less than about 1 wt. %, or less than about 0.5 wt. %.

In some embodiments, two or more ionizing sources can be used, such as two or more electron sources. For example, samples can be treated, in any order, with a beam of electrons, followed by gamma radiation and UV light having wavelengths from about 100 nm to about 280 nm. In some embodiments, samples are treated with three ionizing radiation sources, such as a beam of electrons, gamma radiation, and energetic UV light. The biomass is conveyed through the treatment zone where it can be bombarded with electrons.

It may be advantageous to repeat the treatment to more thoroughly reduce the recalcitrance of the biomass and/or further modify the biomass. In particular the process parameters can be adjusted after a first (e.g., second, third, fourth or more) pass depending on the recalcitrance of the material. In some embodiments, a conveyor can be used which includes a circular system where the biomass is conveyed multiple times through the various processes described above. In some other embodiments multiple treatment devices (e.g., electron beam generators) are used to treat the biomass multiple (e.g., 2, 3, 4 or more) times. In yet other embodiments, a single electron beam generator may be the source of multiple beams (e.g., 2, 3, 4 or more beams) that can be used for treatment of the biomass.

The effectiveness in changing the molecular/supermolecular structure and/or reducing the recalcitrance of the carbohydrate-containing biomass depends on the electron energy used and the dose applied, while exposure time depends on the power and dose. In some embodiments, the dose rate and total dose are adjusted so as not to destroy (e.g., char or burn) the biomass material. For example, the carbohydrates should not be damaged in the processing so that they can be released from the biomass intact, e.g. as monomeric sugars.

In some embodiments, the treatment (with any electron source or a combination of sources) is performed until the material receives a dose of at least about 0.05 Mrad, e.g., at least about 0.1, 0.25, 0.5, 0.75, 1.0, 2.5, 5.0, 7.5, 10.0, 15, 20, 25, 30, 40, 50, 60, 70, 80, 90, 100, 125, 150, 175, or 200 Mrad. In some embodiments, the treatment is performed until the material receives a dose of between 0.1-100 Mrad, 1-200, 5-200, 10-200, 5-150, 50-150 Mrad, 5-100, 5-50, 5-40, 10-50, 10-75, 15-50, 20-35 Mrad.

In some embodiments, relatively low doses of radiation are utilized, e.g., to increase the molecular weight of a cellulosic or lignocellulosic material (with any radiation source or a combination of sources described herein). For example, a dose of at least about 0.05 Mrad, e.g., at least about 0.1 Mrad or at least about 0.25, 0.5, 0.75. 1.0, 1.5, 2.0, 2.5, 3.0, 3.5, 4.0, or at least about 5.0 Mrad. In some embodiments, the irradiation is performed until the material receives a dose of between 0.1 Mrad and 2.0 Mrad, e.g., between 0.5 rad and 4.0 Mrad or between 1.0 Mrad and 3.0 Mrad.

It also can be desirable to irradiate from multiple directions, simultaneously or sequentially, in order to achieve a desired degree of penetration of radiation into the material. For example, depending on the density and moisture content of the material, such as wood, and the type of radiation source used (e.g., gamma or electron beam), the maximum penetration of radiation into the material may be only about 0.75 inch. In such cases, a thicker section (up to 1.5 inch) can be irradiated by first irradiating the material from one side, and then turning the material over and irradiating from the other side. Irradiation from multiple directions can be particularly useful with electron beam radiation, which irradiates faster than gamma radiation but typically does not achieve as great a penetration depth.

### Radiation Opaque Materials

The invention can include processing a material (e.g., lignocellulosic or cellulosic feedstock) in a vault and/or bunker that is constructed using radiation opaque materials. In some implementations, the radiation opaque materials are selected to be capable of shielding the components from X-rays with high energy (short wavelength), which can penetrate many materials. One important factor in designing a radiation shielding enclosure is the attenuation length of the materials used, which will determine the required thickness for a particular material, blend of materials, or layered structure. The attenuation length is the penetration distance at which the radiation is reduced to approximately 1/e (e=Euler's number) times that of the incident radiation. Although virtually all materials are radiation opaque if thick enough, materials containing a high compositional percentage (e.g., density) of elements that have a high Z value (atomic number) have a shorter radiation attenuation length and thus if such materials are used a thinner, lighter shielding can be provided. Examples of high Z value materials that are used in radiation shielding are tantalum and lead. Another important parameter in radiation shielding is the halving distance, which is the thickness of a particular material that will reduce gamma ray intensity by 50%. As an example for X-ray radiation with an energy of 0.1 MeV the halving thickness is about 15.1 mm for concrete and about 2.7 mm for lead, while with an X-ray energy of 1 MeV the halving thickness for concrete is about 44.45 mm and for lead is about 7.9 mm Radiation opaque materials can be materials that are thick or thin so long as they can reduce the radiation that passes through to the other side. Thus, if it is desired that a particular enclosure have a low wall thickness, e.g., for light weight or due to size constraints, the material chosen should have a sufficient Z value and/or attenuation length so that its halving length is less than or equal to the desired wall thickness of the enclosure.

In some cases, the radiation opaque material may be a layered material, for example having a layer of a higher Z value material, to provide good shielding, and a layer of a lower Z value material to provide other properties (e.g., structural integrity, impact resistance, etc.). In some cases, the layered material may be a “graded-Z” laminate, e.g., including a laminate in which the layers provide a gradient from high-Z through successively lower-Z elements. In some cases the radiation opaque materials can be interlocking blocks, for example, lead and/or concrete blocks can be supplied by NELCO Worldwide (Burlington, Mass.), and reconfigurable vaults can be utilized.

A radiation opaque material can reduce the radiation passing through a structure (e.g., a wall, door, ceiling, enclosure, a series of these or combinations of these) formed of the material by about at least about 10%, (e.g., at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%) as compared to the incident radiation. Therefore, an enclosure made of a radiation opaque material can reduce the exposure of equipment/system/components by the same amount. Radiation opaque materials can include stainless steel, metals with Z values above 25 (e.g., lead, iron), concrete, dirt, sand and combinations thereof. Radiation opaque materials can include a barrier in the direction of the incident radiation of at least about 1 mm (e.g., 5 mm, 10 mm, 5 cm, 10 cm, 100 cm, 1 m and even at least about 10 m).

### Radiation Sources

The type of radiation used for treating a feedstock (e.g., a lignocellulosic or cellulosic material) determines the kinds of radiation sources used as well as the radiation devices and associated equipment. The methods, systems and equipment described herein, for example for treating materials with radiation, can utilized sources as described herein as well as any other useful source.

Sources of gamma rays include radioactive nuclei, such as isotopes of cobalt, calcium, technetium, chromium, gallium, indium, iodine, iron, krypton, samarium, selenium, sodium, thallium, and xenon.

Sources of X-rays include electron beam collision with metal targets, such as tungsten or molybdenum or alloys, or compact light sources, such as those produced commercially by Lyncean.

Alpha particles are identical to the nucleus of a helium atom and are produced by the alpha decay of various radioactive nuclei, such as isotopes of bismuth, polonium, astatine, radon, francium, radium, several actinides, such as actinium, thorium, uranium, neptunium, curium, californium, americium, and plutonium.

Sources for ultraviolet radiation include deuterium or cadmium lamps.

Sources for infrared radiation include sapphire, zinc, or selenide window ceramic lamps.

Sources for microwaves include klystrons, Slevin type RF sources, or atom beam sources that employ hydrogen, oxygen, or nitrogen gases.

Accelerators used to accelerate the particles can be electrostatic DC, electrodynamic DC, RF linear, magnetic induction linear or continuous wave. For example, cyclotron type accelerators are available from IBA, Belgium, such as the RHODOTRON™ system, while DC type accelerators are available from RDI, now IBA Industrial, such as the DYNAMITRON®. Ions and ion accelerators are discussed in Introductory Nuclear Physics, Kenneth S. Krane, John Wiley & Sons, Inc. (1988), Krsto Prelec, FIZIKA B 6 (1997) 4, 177-206, Chu, William T., “Overview of Light-Ion Beam Therapy”, Columbus-Ohio, ICRU-IAEA Meeting, 18-20 Mar. 2006, Iwata, Y. et al., “Alternating-Phase-Focused IH-DTL for Heavy-Ion Medical Accelerators”, Proceedings of EPAC 2006, Edinburgh, Scotland, and Leitner, C. M. et al., “Status of the Superconducting ECR Ion Source Venus”, Proceedings of EPAC 2000, Vienna, Austria.

Electrons may be produced by radioactive nuclei that undergo beta decay, such as isotopes of iodine, cesium, technetium, and iridium. Alternatively, an electron gun can be used as an electron source via thermionic emission and accelerated through an accelerating potential. An electron gun generates electrons, which are then accelerated through a large potential (e.g., greater than about 500 thousand, greater than about lmillion, greater than about 2 million, greater than about 5 million, greater than about 6 million, greater than about 7 million, greater than about 8 million, greater than about 9 million, or even greater than 10 million volts) and then scanned magnetically in the x-y plane, where the electrons are initially accelerated in the z direction down the accelerator tube and extracted through a foil window. Scanning the electron beams is useful for increasing the irradiation surface when irradiating materials, e.g., a biomass, that is conveyed through the scanned beam. Scanning the electron beam also distributes the thermal load homogenously on the window and helps reduce the foil window rupture due to local heating by the electron beam. Window foil rupture is a cause of significant down-time due to subsequent necessary repairs and re-starting the electron gun.

Various other irradiating devices may be used in the methods disclosed herein, including field ionization sources, electrostatic ion separators, field ionization generators, thermionic emission sources, microwave discharge ion sources, recirculating or static accelerators, dynamic linear accelerators, van de Graaff accelerators, and folded tandem accelerators. Such devices are disclosed, for example, in U.S. Pat. No. 7,931,784 to Medoff, the complete disclosure of which is incorporated herein by reference.

A beam of electrons can be used as the radiation source. A beam of electrons has the advantages of high dose rates (e.g., 1, 5, or even 10 Mrad per second), high throughput, less containment, and less confinement equipment. Electron beams can also have high electrical efficiency (e.g., 80%), allowing for lower energy usage relative to other radiation methods, which can translate into a lower cost of operation and lower greenhouse gas emissions corresponding to the smaller amount of energy used. Electron beams can be generated, e.g., by electrostatic generators, cascade generators, transformer generators, low energy accelerators with a scanning system, low energy accelerators with a linear cathode, linear accelerators, and pulsed accelerators.

Electrons can also be more efficient at causing changes in the molecular structure of carbohydrate-containing materials, for example, by the mechanism of chain scission. In addition, electrons having energies of 0.5-10 MeV can penetrate low density materials, such as the biomass materials described herein, e.g., materials having a bulk density of less than 0.5 g/cm³, and a depth of 0.3-10 cm. Electrons as an ionizing radiation source can be useful, e.g., for relatively thin piles, layers or beds of materials, e.g., less than about 0.5 inch, e.g., less than about 0.4 inch, 0.3 inch, 0.25 inch, or less than about 0.1 inch. In some embodiments, the energy of each electron of the electron beam is from about 0.3 MeV to about 2.0 MeV (million electron volts), e.g., from about 0.5 MeV to about 1.5 MeV, or from about 0.7 MeV to about 1.25 MeV. Methods of irradiating materials are discussed in U.S. Pat. App. Pub. 2012/0100577 A1, filed Oct. 18, 2011, the entire disclosure of which is herein incorporated by reference.

Electron beam irradiation devices may be procured commercially from Ion Beam Applications, Louvain-la-Neuve, Belgium, NHV Corporation, Japan or the Titan Corporation, San Diego, Calif. Typical electron energies can be 0.5 MeV, 1 MeV, 2 MeV, 4.5 MeV, 7.5 MeV, or 10 MeV. Typical electron beam irradiation device power can be 1 kW, 5 kW, 10 kW, 20 kW, 50 kW, 60 kW, 70 kW, 80 kW, 90 kW, 100 kW, 125 kW, 150 kW, 175 kW, 200 kW, 250 kW, 300 kW, 350 kW, 400 kW, 450 kW, 500 kW, 600 kW, 700 kW, 800 kW, 900 kW or even 1000 kW.

Tradeoffs in considering electron beam irradiation device power specifications include cost to operate, capital costs, depreciation, and device footprint. Tradeoffs in considering exposure dose levels of electron beam irradiation would be energy costs and environment, safety, and health (ESH) concerns. Typically, generators are housed in a vault, e.g., of lead or concrete, especially for production from X-rays that are generated in the process. Tradeoffs in considering electron energies include energy costs.

The electron beam irradiation device can produce either a fixed beam or a scanning beam. A scanning beam may be advantageous with large scan sweep length and high scan speeds, as this would effectively replace a large, fixed beam width. Further, available sweep widths of 0.5 m, 1 m, 2 m or more are available. The scanning beam is preferred in most embodiments describe herein because of the larger scan width and reduced possibility of local heating and failure of the windows.

### Electron Guns—Windows

The extraction system for an electron accelerator can include two window foils. The cooling gas in the two foil window extraction system can be a purge gas or a mixture, for example air, or a pure gas. In one embodiment the gas is an inert gas such as nitrogen, argon, helium and or carbon dioxide. It is preferred to use a gas rather than a liquid since energy losses to the electron beam are minimized Mixtures of pure gas can also be used, either pre-mixed or mixed in line prior to impinging on the windows or in the space between the windows. The cooling gas can be cooled, for example, by using a heat exchange system (e.g., a chiller) and/or by using boil off from a condensed gas (e.g., liquid nitrogen, liquid helium). Window foils are described in PCT/US2013/64332 filed Oct. 10, 2013 the full disclosure of which is incorporated by reference herein.

### Heating and Throughput During Radiation Treatment

Several processes can occur in biomass when electrons from an electron beam interact with matter in inelastic collisions. For example, ionization of the material, chain scission of polymers in the material, cross linking of polymers in the material, oxidation of the material, generation of X-rays (“Bremsstrahlung”) and vibrational excitation of molecules (e.g., phonon generation). Without being bound to a particular mechanism, the reduction in recalcitrance can be due to several of these inelastic collision effects, for example ionization, chain scission of polymers, oxidation and phonon generation. Some of the effects (e.g., especially X-ray generation), necessitate shielding and engineering barriers, for example, enclosing the irradiation processes in a concrete (or other radiation opaque material) vault. Another effect of irradiation, vibrational excitation, is equivalent to heating up the sample. Heating the sample by irradiation can help in recalcitrance reduction, but excessive heating can destroy the material, as will be explained below.

The adiabatic temperature rise (AT) from adsorption of ionizing radiation is given by the equation: ΔT=D/Cp: where D is the average dose in kGy, Cp is the heat capacity in J/g ° C., and ΔT is the change in temperature in ° C. A typical dry biomass material will have a heat capacity close to 2. Wet biomass will have a higher heat capacity dependent on the amount of water since the heat capacity of water is very high (4.19 J/g ° C.). Metals have much lower heat capacities, for example 304 stainless steel has a heat capacity of 0.5 J/g ° C. The temperature change due to the instant adsorption of radiation in a biomass and stainless steel for various doses of radiation is shown in Table 2. In some cases, as indicated in the table, the temperatures are so high that the material decomposes (e.g., is volatilized, carbonized, and/or chared).

TABLE 2
Calculated Temperature increase for biomass and stainless steel.

| Dose (Mrad) | Estimated Biomass ΔT (° C.) | Steel ΔT (° C.) |
|---|---|---|
| 10 | 50 | 200 |
| 50 | 250 (decomposed) | 1000 |
| 100 | 500 (decomposed) | 2000 |
| 150 | 750 (decomposed) | 3000 |
| 200 | 1000 (decomposed) | 4000 |

High temperatures can destroy and or modify the biopolymers in biomass so that the polymers (e.g., cellulose) are unsuitable for further processing. A biomass subjected to high temperatures can become dark, sticky and give off odors indicating decomposition. The stickiness can even make the material hard to convey. The odors can be unpleasant and be a safety issue. In fact, keeping the biomass below about 200° C. has been found to be beneficial in the processes described herein (e.g., below about 190° C., below about 180° C., below about 170° C., below about 160° C., below about 150° C., below about 140° C., below about 130° C., below about 120° C., below about 110° C., between about 60° C. and 180° C., between about 60° C. and 160° C., between about 60° C. and 150° C., between about 60° C. and 140° C., between about 60° C. and 130° C., between about 60° C. and 120° C., between about 80° C. and 180° C., between about 100° C. and 180° C., between about 120° C. and 180° C., between about 140° C. and 180° C., between about 160° C. and 180° C., between about 100° C. and 140° C., between about 80° C. and 120° C.).

It has been found that irradiation above about 10 Mrad is desirable for the processes described herein (e.g., reduction of recalcitrance). A high throughput is also desirable so that the irradiation does not become a bottle neck in processing the biomass. The treatment is governed by a Dose rate equation: M=FP/D·time, where M is the mass of irradiated material (kg), F is the fraction of power that is adsorbed (unit less), P is the emitted power (kW=Voltage in MeV×Current in mA), time is the treatment time (sec) and D is the adsorbed dose (kGy). In an exemplary process where the fraction of adsorbed power is fixed, the Power emitted is constant and a set dosage is desired, the throughput (e.g., M, the biomass processed) can be increased by increasing the irradiation time. However, increasing the irradiation time without allowing the material to cool, can excessively heat the material as exemplified by the calculations shown above. Since biomass has a low thermal conductivity (less than about 0.1 Wm⁻¹K⁻¹), heat dissipation is slow, unlike, for example metals (greater than about 10 Wm⁻¹K⁻¹) which can dissipate energy quickly as long as there is a heat sink to transfer the energy to.

### Electron Guns—Beam Stops

In some embodiments the systems and methods (e.g., that utilize electron beam irradiation to irradiate a lignocellulosic or cellulosic feedstock) include a beam stop (e.g., a shutter). For example, the beam stop can be used to quickly stop or reduce the irradiation of material without powering down the electron beam device. Alternatively the beam stop can be used while powering up the electron beam, e.g., the beam stop can stop the electron beam until a beam current of a desired level is achieved. The beam stop can be placed between the primary foil window and a secondary foil window. For example the beam stop can be mounted so that it is movable, that is, so that it can be moved into and out of the beam path. Even partial coverage of the beam can be used, for example, to control the dose of irradiation. The beam stop can be mounted to the floor, to a conveyor for the biomass, to a wall, to the radiation device (e.g., at the scan horn), or to any structural support. Preferably the beam stop is fixed in relation to the scan horn so that the beam can be effectively controlled by the beam stop. The beam stop can incorporate a hinge, a rail, wheels, slots, or other means allowing for its operation in moving into and out of the beam. The beam stop can be made of any material that will stop at least 5% of the electrons, e.g., at least 10%, 20%, 30%, 40%, 50%, 60%, 70%, at least 80%, 85%, 90%, 91%, 92%, 93%, 94%, 95%, 96%, 97%, 98%, 99% or even about 100% of the electrons.

The beam stop can be made of a metal including, but not limited to, stainless steel, lead, iron, molybdenum, silver, gold, titanium, aluminum, tin, or alloys of these, or laminates (layered materials) made with such metals (e.g., metal-coated ceramic, metal-coated polymer, metal-coated composite, multilayered metal materials).

The beam stop can be cooled, for example, with a cooling fluid such as an aqueous solution or a gas. The beam stop can be partially or completely hollow, for example with cavities. Interior spaces of the beam stop can be used for cooling fluids and gases. The beam stop can be of any shape, including flat, curved, round, oval, square, rectangular, beveled and wedged shapes.

The beam stop can have perforations so as to allow some electrons through, thus controlling (e.g., reducing) the levels of radiation across the whole area of the window, or in specific regions of the window. The beam stop can be a mesh formed, for example, from fibers or wires. Multiple beam stops can be used, together or independently, to control the irradiation. The beam stop can be remotely controlled, e.g., by radio signal or hard wired to a motor for moving the beam into or out of position.

### Beam Dumps

The embodiments disclosed herein (e.g., the utilize ionizing radiation to irradiate a lignocellulosic or cellulosic feedstock) can also include a beam dump when utilizing a radiation treatment. A beam dump's purpose is to safely absorb a beam of charged particles. Like a beam stop, a beam dump can be used to block the beam of charged particles. However, a beam dump is much more robust than a beam stop, and is intended to block the full power of the electron beam for an extended period of time. They are often used to block the beam as the accelerator is powering up.

Beam dumps are also designed to accommodate the heat generated by such beams, and are usually made from materials such as copper, aluminum, carbon, beryllium, tungsten, or mercury. Beam dumps can be cooled, for example, using a cooling fluid that can be in thermal contact with the beam dump.

### Biomass Materials

Lignocellulosic materials (e.g., feedstocks that are saccharified) include, but are not limited to, wood, particle board, forestry wastes (e.g., sawdust, aspen wood, wood chips), grasses, (e.g., switchgrass, miscanthus, cord grass, reed canary grass), grain residues, (e.g., rice hulls, oat hulls, wheat chaff, barley hulls), agricultural waste (e.g., silage, canola straw, wheat straw, barley straw, oat straw, rice straw, jute, hemp, flax, bamboo, sisal, abaca, corn cobs, corn stover, soybean stover, corn fiber, alfalfa, hay, coconut hair), sugar processing residues (e.g., bagasse, beet pulp, agave bagasse), algae, seaweed, manure, sewage, and mixtures of any of these.

In some cases, the lignocellulosic material includes corncobs. Ground or hammermilled corncobs can be spread in a layer of relatively uniform thickness for irradiation, and after irradiation are easy to disperse in the medium for further processing. To facilitate harvest and collection, in some cases the entire corn plant is used, including the corn stalk, corn kernels, and in some cases even the root system of the plant.

Advantageously, no additional nutrients (other than a nitrogen source, e.g., urea or ammonia) are required during fermentation of corncobs or cellulosic or lignocellulosic materials containing significant amounts of corncobs.

Corncobs, before and after comminution, are also easier to convey and disperse, and have a lesser tendency to form explosive mixtures in air than other cellulosic or lignocellulosic materials such as hay and grasses.

Cellulosic materials include, for example, paper, paper products, paper waste, paper pulp, pigmented papers, loaded papers, coated papers, filled papers, magazines, printed matter (e.g., books, catalogs, manuals, labels, calendars, greeting cards, brochures, prospectuses, newsprint), printer paper, polycoated paper, card stock, cardboard, paperboard, materials having a high α-cellulose content such as cotton, and mixtures of any of these. For example paper products as described in U.S. application Ser. No. 13/396,365 (“Magazine Feedstocks” by Medoff et al., filed Feb. 14, 2012), the full disclosure of which is incorporated herein by reference.

Cellulosic materials can also include lignocellulosic materials which have been partially or fully de-lignified.

In some instances other biomass materials can be utilized, for example starchy materials. Starchy materials include starch itself, e.g., corn starch, wheat starch, potato starch or rice starch, a derivative of starch, or a material that includes starch, such as an edible food product or a crop. For example, the starchy material can be arracacha, buckwheat, banana, barley, cassava, kudzu, oca, sago, sorghum, regular household potatoes, sweet potato, taro, yams, or one or more beans, such as favas, lentils or peas. Blends of any two or more starchy materials are also starchy materials. Mixtures of starchy, cellulosic and or lignocellulosic materials can also be used. For example, a biomass can be an entire plant, a part of a plant or different parts of a plant, e.g., a wheat plant, cotton plant, a corn plant, rice plant or a tree. The starchy materials can be treated by any of the methods described herein.

Microbial materials that can be used as feedstock can include, but are not limited to, any naturally occurring or genetically modified microorganism or organism that contains or is capable of providing a source of carbohydrates (e.g., cellulose), for example, protists, e.g., animal protists (e.g., protozoa such as flagellates, amoeboids, ciliates, and sporozoa) and plant protists (e.g., algae such alveolates, chlorarachniophytes, cryptomonads, euglenids, glaucophytes, haptophytes, red algae, stramenopiles, and viridaeplantae). Other examples include seaweed, plankton (e.g., macroplankton, mesoplankton, microplankton, nanoplankton, picoplankton, and femptoplankton), phytoplankton, bacteria (e.g., gram positive bacteria, gram negative bacteria, and extremophiles), yeast and/or mixtures of these. In some instances, microbial biomass can be obtained from natural sources, e.g., the ocean, lakes, bodies of water, e.g., salt water or fresh water, or on land. Alternatively or in addition, microbial biomass can be obtained from culture systems, e.g., large scale dry and wet culture and fermentation systems.

In other embodiments, the biomass materials, such as cellulosic, starchy and lignocellulosic feedstock materials, can be obtained from transgenic microorganisms and plants that have been modified with respect to a wild type variety. Such modifications may be, for example, through the iterative steps of selection and breeding to obtain desired traits in a plant. Furthermore, the plants can have had genetic material removed, modified, silenced and/or added with respect to the wild type variety. For example, genetically modified plants can be produced by recombinant DNA methods, where genetic modifications include introducing or modifying specific genes from parental varieties, or, for example, by using transgenic breeding wherein a specific gene or genes are introduced to a plant from a different species of plant and/or bacteria. Another way to create genetic variation is through mutation breeding wherein new alleles are artificially created from endogenous genes. The artificial genes can be created by a variety of ways including treating the plant or seeds with, for example, chemical mutagens (e.g., using alkylating agents, epoxides, alkaloids, peroxides, formaldehyde), irradiation (e. g., X-rays, gamma rays, neutrons, beta particles, alpha particles, protons, deuterons, UV radiation) and temperature shocking or other external stressing and subsequent selection techniques. Other methods of providing modified genes is through error prone PCR and DNA shuffling followed by insertion of the desired modified DNA into the desired plant or seed. Methods of introducing the desired genetic variation in the seed or plant include, for example, the use of a bacterial carrier, biolistics, calcium phosphate precipitation, electroporation, gene splicing, gene silencing, lipofection, microinjection and viral carriers. Additional genetically modified materials have been described in U.S. application Ser. No. 13/396,369 filed Feb. 14, 2012 the full disclosure of which is incorporated herein by reference.

Any of the methods described herein can be practiced with mixtures of any biomass materials described herein.

### Other Materials

Other materials (e.g., natural or synthetic materials), for example polymers, can be treated and/or made utilizing the methods, equipment and systems described hererin. For example polyethylene (e.g., linear low density ethylene and high density polyethylene), polystyrenes, sulfonated polystyrenes, poly (vinyl chloride), polyesters (e.g., nylons, DACRON™, KODEL™), polyalkylene esters, poly vinyl esters, polyamides (e.g., KEVLAR™), polyethylene terephthalate, cellulose acetate, acetal, poly acrylonitrile, polycarbonates (e.g., LEXAN™), acrylics [e.g., poly (methyl methacrylate), poly(methyl methacrylate), polyacrylonitrile], Poly urethanes, polypropylene, poly butadiene, polyisobutylene, polyacrylonitrile, polychloroprene (e.g. neoprene), poly(cis-1,4-isoprene) [e.g., natural rubber], poly(trans-1,4-isoprene) [e.g., gutta percha], phenol formaldehyde, melamine formaldehyde, epoxides, polyesters, poly amines, polycarboxylic acids, polylactic acids, polyvinyl alcohols, polyanhydrides, poly fluoro carbons (e.g., TEFLON™), silicons (e.g., silicone rubber), polysilanes, poly ethers (e.g., polyethylene oxide, polypropylene oxide), waxes, oils and mixtures of these. Also included are plastics, rubbers, elastomers, fibers, waxes, gels, oils, adhesives, thermoplastics, thermosets, biodegradable polymers, resins made with these polymers, other polymers, other materials and combinations thereof. The polymers can be made by any useful method including cationic polymerization, anionic polymerization, radical polymerization, metathesis polymerization, ring opening polymerization, graft polymerization, addition polymerization. In some cases the treatments disclosed herein can be used, for example, for radically initiated graft polymerization and cross linking. Composites of polymers, for example with glass, metals, biomass (e.g., fibers, particles), ceramics can also be treated and/or made.

Other materials that can be treated by using the methods, systems and equipment disclosed herein are ceramic materials, minerals, metals, inorganic compounds. For example, silicon and germanium crystals, silicon nitrides, metal oxides, semiconductors, insulators, cements and or conductors.

In addition, manufactured multipart or shaped materials (e.g., molded, extruded, welded, riveted, layered or combined in any way) can be treated, for example cables, pipes, boards, enclosures, integrated semiconductor chips, circuit boards, wires, tires, windows, laminated materials, gears, belts, machines, combinations of these. For example, treating a material by the methods described herein can modify the surfaces, for example, making them susceptible to further functionalization, combinations (e.g., welding) and/or treatment can cross link the materials.

### Biomass Material Preparation—Mechanical Treatments

The biomass can be in a dry form, for example with less than about 35% moisture content (e.g., less than about 20%, less than about 15%, less than about 10% less than about 5%, less than about 4%, less than about 3%, less than about 2% or even less than about 1%). The biomass can also be delivered in a wet state, for example as a wet solid, a slurry or a suspension with at least about 10 wt % solids (e.g., at least about 20 wt. %, at least about 30 wt. %, at least about 40 wt. %, at least about 50 wt. %, at least about 60 wt. %, at least about 70 wt. %).

The processes disclosed herein can utilize low bulk density materials, for example cellulosic or lignocellulosic feedstocks that have been physically pretreated to have a bulk density of less than about 0.75 g/cm³, e.g., less than about 0.7, 0.65, 0.60, 0.50, 0.35, 0.25, 0.20, 0.15, 0.10, 0.05 or less, e.g., less than about 0.025 g/cm³. Bulk density is determined using ASTM D1895B. Briefly, the method involves filling a measuring cylinder of known volume with a sample and obtaining a weight of the sample. The bulk density is calculated by dividing the weight of the sample in grams by the known volume of the cylinder in cubic centimeters. If desired, low bulk density materials can be densified, for example, by methods described in U.S. Pat. No. 7,971,809 to Medoff, the full disclosure of which is hereby incorporated by reference.

In some cases, the pre-treatment processing includes screening of the biomass material. Screening can be through a mesh or perforated plate with a desired opening size, for example, less than about 6.35 mm (¼ inch, 0.25 inch), (e.g., less than about 3.18 mm (⅛ inch, 0.125 inch), less than about 1.59 mm ( 1/16 inch, 0.0625 inch), is less than about 0.79 mm ( 1/32 inch, 0.03125 inch), e.g., less than about 0.51 mm ( 1/50 inch, 0.02000 inch), less than about 0.40 mm ( 1/64 inch, 0.015625 inch), less than about 0.23 mm (0.009 inch), less than about 0.20 mm ( 1/128 inch, 0.0078125 inch), less than about 0.18 mm (0.007 inch), less than about 0.13 mm (0.005 inch), or even less than about 0.10 mm ( 1/256 inch, 0.00390625 inch)). In one configuration the desired biomass falls through the perforations or screen and thus biomass larger than the perforations or screen are not irradiated. These larger materials can be re-processed, for example by comminuting, or they can simply be removed from processing. In another configuration material that is larger than the perforations is irradiated and the smaller material is removed by the screening process or recycled. In this kind of a configuration, the conveyor itself (for example a part of the conveyor) can be perforated or made with a mesh. For example, in one particular embodiment the biomass material may be wet and the perforations or mesh allow water to drain away from the biomass before irradiation.

Screening of material can also be by a manual method, for example by an operator or mechanoid (e.g., a robot equipped with a color, reflectivity or other sensor) that removes unwanted material. Screening can also be by magnetic screening wherein a magnet is disposed near the conveyed material and the magnetic material is removed magnetically.

Optional pre-treatment processing can include heating the material. For example a portion of a conveyor conveying the biomass or other material can be sent through a heated zone. The heated zone can be created, for example, by IR radiation, microwaves, combustion (e.g., gas, coal, oil, biomass), resistive heating and/or inductive coils. The heat can be applied from at least one side or more than one side, can be continuous or periodic and can be for only a portion of the material or all the material. For example, a portion of the conveying trough can be heated by use of a heating jacket. Heating can be, for example, for the purpose of drying the material. In the case of drying the material, this can also be facilitated, with or without heating, by the movement of a gas (e.g., air, oxygen, nitrogen, He, CO₂, Argon) over and/or through the biomass as it is being conveyed.

Optionally, pre-treatment processing can include cooling the material. Cooling material is described in U.S. Pat. No. 7,900,857 to Medoff, the disclosure of which in incorporated herein by reference. For example, cooling can be by supplying a cooling fluid, for example water (e.g., with glycerol), or nitrogen (e.g., liquid nitrogen) to the bottom of the conveying trough. Alternatively, a cooling gas, for example, chilled nitrogen can be blown over the biomass materials or under the conveying system.

Another optional pre-treatment processing method can include adding a material to the biomass or other feedstocks. The additional material can be added by, for example, by showering, sprinkling and or pouring the material onto the biomass as it is conveyed. Materials that can be added include, for example, metals, ceramics and/or ions as described in U.S. Pat. App. Pub. 2010/0105119 A1 (filed Oct. 26, 2009) and U.S. Pat. App. Pub. 2010/0159569 A1 (filed Dec. 16, 2009), the entire disclosures of which are incorporated herein by reference. Optional materials that can be added include acids and bases. Other materials that can be added are oxidants (e.g., peroxides, chlorates), polymers, polymerizable monomers (e.g., containing unsaturated bonds), water, catalysts, enzymes and/or organisms. Materials can be added, for example, in pure form, as a solution in a solvent (e.g., water or an organic solvent) and/or as a solution. In some cases the solvent is volatile and can be made to evaporate e.g., by heating and/or blowing gas as previously described. The added material may form a uniform coating on the biomass or be a homogeneous mixture of different components (e.g., biomass and additional material). The added material can modulate the subsequent irradiation step by increasing the efficiency of the irradiation, damping the irradiation or changing the effect of the irradiation (e.g., from electron beams to X-rays or heat). The method may have no impact on the irradiation but may be useful for further downstream processing. The added material may help in conveying the material, for example, by lowering dust levels.

Biomass can be delivered to a conveyor (e.g., vibratory conveyors used in the vaults herein described) by a belt conveyor, a pneumatic conveyor, a screw conveyor, a hopper, a pipe, manually or by a combination of these. The biomass can, for example, be dropped, poured and/or placed onto the conveyor by any of these methods. In some embodiments the material is delivered to the conveyor using an enclosed material distribution system to help maintain a low oxygen atmosphere and/or control dust and fines. Lofted or air suspended biomass fines and dust are undesirable because these can form an explosion hazard or damage the window foils of an electron gun (if such a device is used for treating the material).

The material can be leveled to form a uniform thickness between about 0.0312 and 5 inches (e.g., between about 0.0625 and 2.000 inches, between about 0.125 and 1 inches, between about 0.125 and 0.5 inches, between about 0.3 and 0.9 inches, between about 0.2 and 0.5 inches between about 0.25 and 1.0 inches, between about 0.25 and 0.5 inches, 0.100+/−0.025 inches, 0.150+/−0.025 inches, 0.200+/−0.025 inches, 0.250+/−0.025 inches, 0.300+/−0.025 inches, 0.350+/−0.025 inches, 0.400+/−0.025 inches, 0.450+/−0.025 inches, 0.500+/−0.025 inches, 0.550+/−0.025 inches, 0.600+/−0.025 inches, 0.700+/−0.025 inches, 0.750+/−0.025 inches, 0.800+/−0.025 inches, 0.850+/−0.025 inches, 0.900+/−0.025 inches, 0.900+/−0.025 inches.

Generally, it is preferred to convey the material as quickly as possible through the electron beam to maximize throughput. For example the material can be conveyed at rates of at least 1 ft/min, e.g., at least 2 ft/min, at least 3 ft/min, at least 4 ft/min, at least 5 ft/min, at least 10 ft/min, at least 15 ft/min, 20, 25, 30, 35, 40, 45, 50 ft/min. The rate of conveying is related to the beam current, for example, for a ¼ inch thick biomass and 100 mA, the conveyor can move at about 20 ft/min to provide a useful irradiation dosage, at 50 mA the conveyor can move at about 10 ft/min to provide approximately the same irradiation dosage.

After the biomass material has been conveyed through the radiation zone, optional post-treatment processing can be done. The optional post-treatment processing can, for example, be a process described with respect to the pre-irradiation processing. For example, the biomass can be screened, heated, cooled, and/or combined with additives. Uniquely to post-irradiation, quenching of the radicals can occur, for example, quenching of radicals by the addition of fluids or gases (e.g., oxygen, nitrous oxide, ammonia, liquids), using pressure, heat, and/or the addition of radical scavengers. For example, the biomass can be conveyed out of the enclosed conveyor and exposed to a gas (e.g., oxygen) where it is quenched, forming carboxylated groups. In one embodiment the biomass is exposed during irradiation to the reactive gas or fluid. Quenching of biomass that has been irradiated is described in U.S. Pat. No. 8,083,906 to Medoff, the entire disclosure of which is incorporate herein by reference.

If desired, one or more mechanical treatments can be used in addition to irradiation to further reduce the recalcitrance of the carbohydrate-containing material. These processes can be applied before, during and or after irradiation.

In some cases, the mechanical treatment may include an initial preparation of the feedstock as received, e.g., size reduction of materials, such as by comminution, e.g., cutting, grinding, shearing, pulverizing or chopping. For example, in some cases, loose feedstock (e.g., recycled paper, starchy materials, or switchgrass) is prepared by shearing or shredding. Mechanical treatment may reduce the bulk density of the carbohydrate-containing material, increase the surface area of the carbohydrate-containing material and/or decrease one or more dimensions of the carbohydrate-containing material.

Alternatively, or in addition, the feedstock material can be treated with another treatment, for example chemical treatments, such as an with an acid (HCl, H₂SO₄, H₃PO₄), a base (e.g., KOH and NaOH), a chemical oxidant (e.g., peroxides, chlorates, ozone), irradiation, steam explosion, pyrolysis, sonication, oxidation, chemical treatment. The treatments can be in any order and in any sequence and combinations. For example, the feedstock material can first be physically treated by one or more treatment methods, e.g., chemical treatment including and in combination with acid hydrolysis (e.g., utilizing HCl, H₂SO₄, H₃PO₄), radiation, sonication, oxidation, pyrolysis or steam explosion, and then mechanically treated. This sequence can be advantageous since materials treated by one or more of the other treatments, e.g., irradiation or pyrolysis, tend to be more brittle and, therefore, it may be easier to further change the structure of the material by mechanical treatment. As another example, a feedstock material can be conveyed through ionizing radiation using a conveyor as described herein and then mechanically treated. Chemical treatment can remove some or all of the lignin (for example chemical pulping) and can partially or completely hydrolyze the material. The methods also can be used with pre-hydrolyzed material. The methods also can be used with material that has not been pre hydrolyzed The methods can be used with mixtures of hydrolyzed and non-hydrolyzed materials, for example with about 50% or more non-hydrolyzed material, with about 60% or more non-hydrolyzed material, with about 70% or more non-hydrolyzed material, with about 80% or more non-hydrolyzed material or even with 90% or more non-hydrolyzed material.

In addition to size reduction, which can be performed initially and/or later in processing, mechanical treatment can also be advantageous for “opening up,” “stressing,” breaking or shattering the carbohydrate-containing materials, making the cellulose of the materials more susceptible to chain scission and/or disruption of crystalline structure during the physical treatment.

Methods of mechanically treating the carbohydrate-containing material include, for example, milling or grinding. Milling may be performed using, for example, a hammer mill, ball mill, colloid mill, conical or cone mill, disk mill, edge mill, Wiley mill, grist mill or other mill. Grinding may be performed using, for example, a cutting/impact type grinder. Some exemplary grinders include stone grinders, pin grinders, coffee grinders, and burr grinders. Grinding or milling may be provided, for example, by a reciprocating pin or other element, as is the case in a pin mill Other mechanical treatment methods include mechanical ripping or tearing, other methods that apply pressure to the fibers, and air attrition milling. Suitable mechanical treatments further include any other technique that continues the disruption of the internal structure of the material that was initiated by the previous processing steps.

Mechanical feed preparation systems can be configured to produce streams with specific characteristics such as, for example, specific maximum sizes, specific length-to-width, or specific surface areas ratios. Physical preparation can increase the rate of reactions, improve the movement of material on a conveyor, improve the irradiation profile of the material, improve the radiation uniformity of the material, or reduce the processing time required by opening up the materials and making them more accessible to processes and/or reagents, such as reagents in a solution.

The bulk density of feedstocks can be controlled (e.g., increased). In some situations, it can be desirable to prepare a low bulk density material, e.g., by densifying the material (e.g., densification can make it easier and less costly to transport to another site) and then reverting the material to a lower bulk density state (e.g., after transport). The material can be densified, for example from less than about 0.2 g/cc to more than about 0.9 g/cc (e.g., less than about 0.3 to more than about 0.5 g/cc, less than about 0.3 to more than about 0.9 g/cc, less than about 0.5 to more than about 0.9 g/cc, less than about 0.3 to more than about 0.8 g/cc, less than about 0.2 to more than about 0.5 g/cc). For example, the material can be densified by the methods and equipment disclosed in U.S. Pat. No. 7,932,065 to Medoff and International Publication No. WO 2008/073186 (which was filed Oct. 26, 2007, was published in English, and which designated the United States), the full disclosures of which are incorporated herein by reference. Densified materials can be processed by any of the methods described herein, or any material processed by any of the methods described herein can be subsequently densified.

In some embodiments, the material to be processed is in the form of a fibrous material that includes fibers provided by shearing a fiber source. For example, the shearing can be performed with a rotary knife cutter.

For example, a fiber source, e.g., that is recalcitrant or that has had its recalcitrance level reduced, can be sheared, e.g., in a rotary knife cutter, to provide a first fibrous material. The first fibrous material is passed through a first screen, e.g., having an average opening size of 1.59 mm or less ( 1/16 inch, 0.0625 inch), provide a second fibrous material. If desired, the fiber source can be cut prior to the shearing, e.g., with a shredder. For example, when a paper is used as the fiber source, the paper can be first cut into strips that are, e.g., ¼- to ½-inch wide, using a shredder, e.g., a counter-rotating screw shredder, such as those manufactured by Munson (Utica, N.Y.). As an alternative to shredding, the paper can be reduced in size by cutting to a desired size using a guillotine cutter. For example, the guillotine cutter can be used to cut the paper into sheets that are, e.g., 10 inches wide by 12 inches long.

In some embodiments, the shearing of the fiber source and the passing of the resulting first fibrous material through a first screen are performed concurrently. The shearing and the passing can also be performed in a batch-type process.

For example, a rotary knife cutter can be used to concurrently shear the fiber source and screen the first fibrous material. A rotary knife cutter includes a hopper that can be loaded with a shredded fiber source prepared by shredding a fiber source.

In some implementations, the feedstock is physically treated prior to saccharification and/or fermentation. Physical treatment processes can include one or more of any of those described herein, such as mechanical treatment, chemical treatment, irradiation, sonication, oxidation, pyrolysis or steam explosion. Treatment methods can be used in combinations of two, three, four, or even all of these technologies (in any order). When more than one treatment method is used, the methods can be applied at the same time or at different times. Other processes that change a molecular structure of a biomass feedstock may also be used, alone or in combination with the processes disclosed herein.

Mechanical treatments that may be used, and the characteristics of the mechanically treated carbohydrate-containing materials, are described in further detail in U.S. Pat. App. Pub. 2012/0100577 A1, filed Oct. 18, 2011, the full disclosure of which is hereby incorporated herein by reference.

### Sonication, Pyrolysis, Oxidation, Steam Explosion

If desired, one or more sonication, pyrolysis, oxidative, or steam explosion processes can be used instead of or in addition to irradiation to reduce or further reduce the recalcitrance of the carbohydrate-containing material. For example, these processes can be applied before, during and or after irradiation. These processes are described in detail in U.S. Pat. No. 7,932,065 to Medoff, the full disclosure of which is incorporated herein by reference.

### Intermediates and Products

Using the processes described herein, the biomass material can be converted to one or more products, such as energy, fuels, foods and materials. For example, intermediates and products such as organic acids, salts of organic acids, anhydrides, esters of organic acids and fuels, e.g., fuels for internal combustion engines or feedstocks for fuel cells. Systems and processes are described herein that can use as feedstock cellulosic and/or lignocellulosic materials that are readily available, but often can be difficult to process, e.g., municipal waste streams and waste paper streams, such as streams that include newspaper, Kraft paper, corrugated paper or mixtures of these.

Specific examples of products include, but are not limited to, hydrogen, sugars (e.g., glucose, xylose, arabinose, mannose, galactose, fructose, disaccharides, oligosaccharides and polysaccharides), alcohols (e.g., monohydric alcohols or dihydric alcohols, such as ethanol, n-propanol, isobutanol, sec-butanol, tert-butanol or n-butanol), hydrated or hydrous alcohols (e.g., containing greater than 10%, 20%, 30% or even greater than 40% water), biodiesel, organic acids, hydrocarbons (e.g., methane, ethane, propane, isobutene, pentane, n-hexane, biodiesel, bio-gasoline and mixtures thereof), co-products (e.g., proteins, such as cellulolytic proteins (enzymes) or single cell proteins), and mixtures of any of these in any combination or relative concentration, and optionally in combination with any additives (e.g., fuel additives). Other examples include carboxylic acids, salts of a carboxylic acid, a mixture of carboxylic acids and salts of carboxylic acids and esters of carboxylic acids (e.g., methyl, ethyl and n-propyl esters), ketones (e.g., acetone), aldehydes (e.g., acetaldehyde), alpha and beta unsaturated acids (e.g., acrylic acid) and olefins (e.g., ethylene). Other alcohols and alcohol derivatives include propanol, propylene glycol, 1,4-butanediol, 1,3-propanediol, sugar alcohols (e.g., erythritol, glycol, glycerol, sorbitol threitol, arabitol, ribitol, mannitol, dulcitol, fucitol, iditol, isomalt, maltitol, lactitol, xylitol and other polyols), and methyl or ethyl esters of any of these alcohols. Other products include methyl acrylate, methylmethacrylate, lactic acid, citric acid, formic acid, acetic acid, propionic acid, butyric acid, succinic acid, valeric acid, caproic acid, 3-hydroxypropionic acid, palmitic acid, stearic acid, oxalic acid, malonic acid, glutaric acid, oleic acid, linoleic acid, glycolic acid, gamma-hydroxybutyric acid, and mixtures thereof, salts of any of these acids, mixtures of any of the acids and their respective salts.

Any combination of the above products with each other, and/or of the above products with other products, which other products may be made by the processes described herein or otherwise, may be packaged together and sold as products. The products may be combined, e.g., mixed, blended or co-dissolved, or may simply be packaged or sold together.

Any of the products or combinations of products described herein may be sanitized or sterilized prior to selling the products, e.g., after purification or isolation or even after packaging, to neutralize one or more potentially undesirable contaminants that could be present in the product(s). Such sanitation can be done with electron bombardment, for example, be at a dosage of less than about 20 Mrad, e.g., from about 0.1 to 15 Mrad, from about 0.5 to 7 Mrad, or from about 1 to 3 Mrad.

The processes described herein can produce various by-product streams useful for generating steam and electricity to be used in other parts of the plant (co-generation) or sold on the open market. For example, steam generated from burning by-product streams can be used in a distillation process. As another example, electricity generated from burning by-product streams can be used to power electron beam generators used in pretreatment.

The by-products used to generate steam and electricity are derived from a number of sources throughout the process. For example, anaerobic digestion of wastewater can produce a biogas high in methane and a small amount of waste biomass (sludge). As another example, post-saccharification and/or post-distillate solids (e.g., unconverted lignin, cellulose, and hemicellulose remaining from the pretreatment and primary processes) can be used, e.g., burned, as a fuel.

Other intermediates and products, including food and pharmaceutical products, are described in U.S. Pat. App. Pub. 2010/0124583 A1, published May 20, 2010, to Medoff, the full disclosure of which is hereby incorporated by reference herein.

### Lignin Derived Products

The spent biomass (e.g., spent lignocellulosic material) from lignocellulosic processing by the methods described are expected to have a high lignin content and in addition to being useful for producing energy through combustion in a Co-Generation plant, may have uses as other valuable products. For example, the lignin can be used as captured as a plastic, or it can be synthetically upgraded to other plastics. In some instances, it can also be converted to lignosulfonates, which can be utilized as binders, dispersants, emulsifiers or sequestrants.

When used as a binder, the lignin or a lignosulfonate can, e.g., be utilized in coal briquettes, in ceramics, for binding carbon black, for binding fertilizers and herbicides, as a dust suppressant, in the making of plywood and particle board, for binding animal feeds, as a binder for fiberglass, as a binder in linoleum paste and as a soil stabilizer.

When used as a dispersant, the lignin or lignosulfonates can be used, for example in, concrete mixes, clay and ceramics, dyes and pigments, leather tanning and in gypsum board.

When used as an emulsifier, the lignin or lignosulfonates can be used, e.g., in asphalt, pigments and dyes, pesticides and wax emulsions.

As a sequestrant, the lignin or lignosulfonates can be used, e.g., in micro-nutrient systems, cleaning compounds and water treatment systems, e.g., for boiler and cooling systems.

For energy production lignin generally has a higher energy content than holocellulose (cellulose and hemicellulose) since it contains more carbon than homocellulose. For example, dry lignin can have an energy content of between about 11,000 and 12,500 BTU per pound, compared to 7,000 an 8,000 BTU per pound of holocellulose. As such, lignin can be densified and converted into briquettes and pellets for burning. For example, the lignin can be converted into pellets by any method described herein. For a slower burning pellet or briquette, the lignin can be crosslinked, such as applying a radiation dose of between about 0.5 Mrad and 5 Mrad. Crosslinking can make a slower burning form factor. The form factor, such as a pellet or briquette, can be converted to a “synthetic coal” or charcoal by pyrolyzing in the absence of air, e.g., at between 400 and 950° C. Prior to pyrolyzing, it can be desirable to crosslink the lignin to maintain structural integrity.

### Saccharification

In order to convert the feedstock to a form that can be readily processed, the glucan- or xylan-containing cellulose in the feedstock can be hydrolyzed to low molecular weight carbohydrates, such as sugars, by a saccharifying agent, e.g., an enzyme or acid, a process referred to as saccharification. The low molecular weight carbohydrates can then be used, for example, in an existing manufacturing plant, such as a single cell protein plant, an enzyme manufacturing plant, or a fuel plant, e.g., an ethanol manufacturing facility.

The feedstock can be hydrolyzed using an enzyme, e.g., by combining the materials and the enzyme in a solvent, e.g., in an aqueous solution.

Alternatively, the enzymes can be supplied by organisms that break down biomass, such as the cellulose and/or the lignin portions of the biomass, contain or manufacture various cellulolytic enzymes (cellulases), ligninases or various small molecule biomass-degrading metabolites. These enzymes may be a complex of enzymes that act synergistically to degrade crystalline cellulose or the lignin portions of biomass. Examples of cellulolytic enzymes include: endoglucanases, cellobiohydrolases, and cellobiases (beta-glucosidases).

During saccharification a cellulosic substrate can be initially hydrolyzed by endoglucanases at random locations producing oligomeric intermediates. These intermediates are then substrates for exo-splitting glucanases such as cellobiohydrolase to produce cellobiose from the ends of the cellulose polymer. Cellobiose is a water-soluble 1,4-linked dimer of glucose. Finally, cellobiase cleaves cellobiose to yield glucose. The efficiency (e.g., time to hydrolyze and/or completeness of hydrolysis) of this process depends on the recalcitrance of the cellulosic material.

Therefore, the treated biomass materials can be saccharified, generally by combining the material and a cellulase enzyme in a fluid medium, e.g., an aqueous solution. In some cases, the material is boiled, steeped, or cooked in hot water prior to saccharification, as described in U.S. Pat. App. Pub. 2012/0100577 A1 by Medoff and Masterman, published on Apr. 26, 2012, the entire contents of which are incorporated herein.

The saccharification process can be partially or completely performed in a tank (e.g., a tank having a volume of at least 4000, 40,000, or 500,000 L) in a manufacturing plant, and/or can be partially or completely performed in transit, e.g., in a rail car, tanker truck, or in a supertanker or the hold of a ship. The time required for complete saccharification will depend on the process conditions and the carbohydrate-containing material and enzyme used. If saccharification is performed in a manufacturing plant under controlled conditions, the cellulose may be substantially entirely converted to sugar, e.g., glucose in about 12-96 hours. If saccharification is performed partially or completely in transit, saccharification may take longer.

It is generally preferred that the tank contents be mixed during saccharification, e.g., using jet mixing as described in International App. No. PCT/US2010/035331, filed May 18, 2010, which was published in English as WO 2010/135380 and designated the United States, the full disclosure of which is incorporated by reference herein.

The addition of surfactants can enhance the rate of saccharification. Examples of surfactants include non-ionic surfactants, such as a Tween® 20 or Tween® 80 polyethylene glycol surfactants, ionic surfactants, or amphoteric surfactants.

It is generally preferred that the concentration of the sugar solution resulting from saccharification be relatively high, e.g., greater than 40%, or greater than 50, 60, 70, 80, 90 or even greater than 95% by weight. Water may be removed, e.g., by evaporation, to increase the concentration of the sugar solution. This reduces the volume to be shipped, and also inhibits microbial growth in the solution.

Alternatively, sugar solutions of lower concentrations may be used, in which case it may be desirable to add an antimicrobial additive, e.g., a broad spectrum antibiotic, in a low concentration, e.g., 50 to 150 ppm. Other suitable antibiotics include amphotericin B, ampicillin, chloramphenicol, ciprofloxacin, gentamicin, hygromycin B, kanamycin, neomycin, penicillin, puromycin, streptomycin. Antibiotics will inhibit growth of microorganisms during transport and storage, and can be used at appropriate concentrations, e.g., between 15 and 1000 ppm by weight, e.g., between 25 and 500 ppm, or between 50 and 150 ppm. If desired, an antibiotic can be included even if the sugar concentration is relatively high. Alternatively, other additives with anti-microbial of preservative properties may be used. Preferably the antimicrobial additive(s) are food-grade.

A relatively high concentration solution can be obtained by limiting the amount of water added to the carbohydrate-containing material with the enzyme. The concentration can be controlled, e.g., by controlling how much saccharification takes place. For example, concentration can be increased by adding more carbohydrate-containing material to the solution. In order to keep the sugar that is being produced in solution, a surfactant can be added, e.g., one of those discussed above. Solubility can also be increased by increasing the temperature of the solution. For example, the solution can be maintained at a temperature of 40-50° C., 60-80° C., or even higher.

### Saccharifying Agents

Suitable cellulolytic enzymes include cellulases from species in the genera Bacillus, Coprinus, Myceliophthora, Cephalosporium, Scytalidium, Penicillium, Aspergillus, Pseudomonas, Humicola, Fusarium, Thielavia, Acremonium, Chrysosporium and Trichoderma, especially those produced by a strain selected from the species Aspergillus (see, e.g., EP Pub. No. 0 458 162), Humicola insolens (reclassified as Scytalidium thermophilum, see, e.g., U.S. Pat. No. 4,435,307), Coprinus cinereus, Fusarium oxysporum, Myceliophthora thermophila, Meripilus giganteus, Thielavia terrestris, Acremonium sp. (including, but not limited to, A. persicinum, A. acremonium, A. brachypenium, A. dichromosporum, A. obclavatum, A. pinkertoniae, A. roseogriseum, A. incoloratum, and A. furatum). Preferred strains include Humicola insolens DSM 1800, Fusarium oxysporum DSM 2672, Myceliophthora thermophila CBS 117.65, Cephalosporium sp. RYM-202, Acremonium sp. CBS 478.94, Acremonium sp. CBS 265.95, Acremonium persicinum CBS 169.65, Acremonium acremonium AHU 9519, Cephalosporium sp. CBS 535.71, Acremonium brachypenium CBS 866.73, Acremonium dichromosporum CBS 683.73, Acremonium obclavatum CBS 311.74, Acremonium pinkertoniae CBS 157.70, Acremonium roseogriseum CBS 134.56, Acremonium incoloratum CBS 146.62, and Acremonium furatum CBS 299.70H. Cellulolytic enzymes may also be obtained from Chrysosporium, preferably a strain of Chrysosporium lucknowense. Additional strains that can be used include, but are not limited to, Trichoderma (particularly T. viride, T. reesei, and T. koningii), alkalophilic Bacillus (see, for example, U.S. Pat. No. 3,844,890 and EP Pub. No. 0 458 162), and Streptomyces (see, e.g., EP Pub. No. 0 458 162).

In addition to or in combination to enzymes, acids, bases and other chemicals (e.g., oxidants) can be utilized to saccharify lignocellulosic and cellulosic materials. These can be used in any combination or sequence (e.g., before, after and/or during addition of an enzyme). For example strong mineral acids can be utilized (e.g. HCl, H₂SO₄, H₃PO₄) and strong bases (e.g., NaOH, KOH).

### Sugars

In the processes described herein, for example after saccharification, sugars (e.g., glucose and xylose) can be isolated. For example sugars can be isolated by precipitation, crystallization, chromatography (e.g., simulated moving bed chromatography, high pressure chromatography), centrifugation, extraction, any other isolation method known in the art, and combinations thereof.

### Hydrogenation and Other Chemical Transformations

The processes described herein can include hydrogenation. For example glucose and xylose can be hydrogenated to sorbitol and xylitol respectively. Hydrogenation can be accomplished by use of a catalyst (e.g., Pt/gamma-Al₂O₃, Ru/C, Raney Nickel, or other catalysts know in the art) in combination with H₂ under high pressure (e.g., 10 to 12000 psi, between about 100 and 10,000 psi). Other types of chemical transformation of the products from the processes described herein can be used, for example production of organic sugar derived products such (e.g., furfural and furfural-derived products). Chemical transformations of sugar derived products are described in U.S. Ser. No. 13/934,704 filed Jul. 3, 2013, the entire disclosure of which is incorporated herein by reference in its entirety.

### Fermentation

Yeast and Zymomonas bacteria, for example, can be used for fermentation or conversion of sugar(s) to alcohol(s). Other microorganisms are discussed below. The optimum pH for fermentations is about pH 4 to 7. For example, the optimum pH for yeast is from about pH 4 to 5, while the optimum pH for Zymomonas is from about pH 5 to 6. Typical fermentation times are about 24 to 168 hours (e.g., 24 to 96 hrs) with temperatures in the range of 20° C. to 40° C. (e.g., 26° C. to 40° C.), however thermophilic microorganisms prefer higher temperatures.

In some embodiments, e.g., when anaerobic organisms are used, at least a portion of the fermentation is conducted in the absence of oxygen, e.g., under a blanket of an inert gas such as N₂, Ar, He, CO₂ or mixtures thereof. Additionally, the mixture may have a constant purge of an inert gas flowing through the tank during part of or all of the fermentation. In some cases, anaerobic conditions can be achieved or maintained by carbon dioxide production during the fermentation and no additional inert gas is needed.

In some embodiments, all or a portion of the fermentation process can be interrupted before the low molecular weight sugar is completely converted to a product (e.g., ethanol). The intermediate fermentation products include sugar and carbohydrates in high concentrations. The sugars and carbohydrates can be isolated via any means known in the art. These intermediate fermentation products can be used in preparation of food for human or animal consumption. Additionally or alternatively, the intermediate fermentation products can be ground to a fine particle size in a stainless-steel laboratory mill to produce a flour-like substance. Jet mixing may be used during fermentation, and in some cases saccharification and fermentation are performed in the same tank.

Nutrients for the microorganisms may be added during saccharification and/or fermentation, for example the food-based nutrient packages described in U.S. Pat. App. Pub. 2012/0052536, filed Jul. 15, 2011, the complete disclosure of which is incorporated herein by reference.

“Fermentation” includes the methods and products that are disclosed in applications No. PCT/US2012/71093 published Jun. 27, 2013, PCT/US2012/71907 published Jun. 27, 2012, and PCT/US2012/71083 published Jun. 27, 2012 the contents of which are incorporated by reference herein in their entirety.

Mobile fermenters can be utilized, as described in International App. No. PCT/US2007/074028 (which was filed Jul. 20, 2007, was published in English as WO 2008/011598 and designated the United States) and has a US issued U.S. Pat. No. 8,318,453, the contents of which are incorporated herein in its entirety. Similarly, the saccharification equipment can be mobile. Further, saccharification and/or fermentation may be performed in part or entirely during transit.

### Fermentation Agents

The microorganism(s) used in fermentation can be naturally-occurring microorganisms and/or engineered microorganisms. For example, the microorganism can be a bacterium (including, but not limited to, e.g., a cellulolytic bacterium), a fungus, (including, but not limited to, e.g., a yeast), a plant, a protist, e.g., a protozoa or a fungus-like protest (including, but not limited to, e.g., a slime mold), or an alga. When the organisms are compatible, mixtures of organisms can be utilized.

Suitable fermenting microorganisms have the ability to convert carbohydrates, such as glucose, fructose, xylose, arabinose, mannose, galactose, oligosaccharides or polysaccharides into fermentation products. Fermenting microorganisms include strains of the genus Saccharomyces spp. (including, but not limited to, S. cerevisiae (baker's yeast), S. distaticus, S. uvarum), the genus Kluyveromyces, (including, but not limited to, K. marxianus, K fragilis), the genus Candida (including, but not limited to, C. pseudotropicalis, and C. brassicae), Pichia stipitis (a relative of Candida shehatae), the genus Clavispora (including, but not limited to, C. lusitaniae and C. opuntiae), the genus Pachysolen (including, but not limited to, P. tannophilus), the genus Bretannomyces (including, but not limited to, e.g., B. clausenii (Philippidis, G. P., 1996, Cellulose bioconversion technology, in Handbook on Bioethanol: Production and Utilization, Wyman, C.E., ed., Taylor & Francis, Washington, D.C., 179-212)). Other suitable microorganisms include, for example, Zymomonas mobilis, Clostridium spp. (including, but not limited to, C. thermocellum (Philippidis, 1996, supra), C. saccharobutylacetonicum, C. tyrobutyricum C. saccharobutylicum, C. Puniceum, C. beijernckii, and C. acetobutylicum), Moniliella spp. (including but not limited to M. pollinis, M. tomentosa, M. madida, M. nigrescens, M oedocephali, M. megachiliensis), Yarrowia lipolytica, Aureobasidium sp., Trichosporonoides sp., Trigonopsis variabilis, Trichosporon sp., Moniliellaacetoabutans sp., Typhula variabilis, Candida magnoliae, Ustilaginomycetes sp., Pseudozyma tsukubaensis, yeast species of genera Zygosaccharomyces, Debaryomyces, Hansenula and Pichia, and fungi of the dematioid genus Torula (e.g., T. corallina).

Many such microbial strains are publicly available, either commercially or through depositories such as the ATCC (American Type Culture Collection, Manassas, Va., USA), the NRRL (Agricultural Research Service Culture Collection, Peoria, Ill., USA), or the DSMZ (Deutsche Sammlung von Mikroorganismen and Zellkulturen GmbH, Braunschweig, Germany), to name a few.

Commercially available yeasts include, for example, RED STAR®/Lesaffre Ethanol Red (available from Red Star/Lesaffre, USA), FALI® (available from Fleischmann's Yeast, a division of Burns Philip Food Inc., USA), SUPERSTART® (available from Alltech, now Lalemand), GERT STRAND® (available from Gert Strand AB, Sweden) and FERMOL® (available from DSM Specialties).

### Distillation

After fermentation, the resulting fluids can be distilled using, for example, a “beer column” to separate ethanol and other alcohols from the majority of water and residual solids. The distillation can be done under vacuum (e.g., to reduce decomposition of products in the solution such as sugars) The vapor exiting the beer column can beat least 35% by weight (e.g., at least 40%, at least 50% or at least 90% by weight) ethanol and can be fed to a rectification column A mixture of nearly azeotropic (e.g., at least about 92.5% ethanol and water from the rectification column can be purified to pure (e.g., at least about 99.5% or even about 100%) ethanol using vapor-phase molecular sieves. The beer column bottoms can be sent to the first effect of a three-effect evaporator. The rectification column reflux condenser can provide heat for this first effect. After the first effect, solids can be separated using a centrifuge and dried in a rotary dryer. A portion (25%) of the centrifuge effluent can be recycled to fermentation and the rest sent to the second and third evaporator effects. Most of the evaporator condensate can be returned to the process as fairly clean condensate with a small portion split off to waste water treatment to prevent build-up of low-boiling compounds.

### Hydrocarbon-Containing Materials

In other embodiments utilizing the methods and systems described herein, hydrocarbon-containing materials can be processed. Any process described herein can be used to treat any hydrocarbon-containing material herein described. “Hydrocarbon-containing materials,” as used herein, is meant to include oil sands, oil shale, tar sands, coal dust, coal slurry, bitumen, various types of coal, and other naturally-occurring and synthetic materials that include both hydrocarbon components and solid matter. The solid matter can include rock, sand, clay, stone, silt, drilling slurry, or other solid organic and/or inorganic matter. The term can also include waste products such as drilling waste and by-products, refining waste and by-products, or other waste products containing hydrocarbon components, such as asphalt shingling and covering, asphalt pavement, etc.

In yet other embodiments utilizing the methods and systems described herein, wood and wood containing produces can be processed. For example lumber products can be processed, e.g. boards, sheets, laminates, beams, particle boards, composites, rough cut wood, soft wood and hard wood. In addition cut trees, bushes, wood chips, saw dust, roots, bark, stumps, decomposed wood and other wood containing biomass material can be processed.

### Conveying Systems

Various conveying systems can be used to convey the biomass material, for example, to a vault and under an electron beam in a vault. Exemplary conveyors are belt conveyors, pneumatic conveyors, screw conveyors, carts, trains, trains or carts on rails, elevators, front loaders, backhoes, cranes, various scrapers and shovels, trucks, and throwing devices can be used. For example, vibratory conveyors can be used in various processes described herein. Vibratory conveyors are described in PCT/US2013/64289 filed Oct. 10, 2013 the full disclosure of which is incorporated by reference herein.

Optionally, one or more conveying systems can be enclosed. When using an enclosure, the enclosed conveyor can also be purged with an inert gas so as to maintain an atmosphere at a reduced oxygen level. Keeping oxygen levels low avoids the formation of ozone which in some instances is undesirable due to its reactive and toxic nature. For example the oxygen can be less than about 20% (e.g., less than about 10%, less than about 1%, less than about 0.1%, less than about 0.01%, or even less than about 0.001% oxygen). Purging can be done with an inert gas including, but not limited to, nitrogen, argon, helium or carbon dioxide. This can be supplied, for example, from a boil off of a liquid source (e.g., liquid nitrogen or helium), generated or separated from air in situ, or supplied from tanks. The inert gas can be recirculated and any residual oxygen can be removed using a catalyst, such as a copper catalyst bed. Alternatively, combinations of purging, recirculating and oxygen removal can be done to keep the oxygen levels low.

The enclosed conveyor can also be purged with a reactive gas that can react with the biomass. This can be done before, during or after the irradiation process. The reactive gas can be, but is not limited to, nitrous oxide, ammonia, oxygen, ozone, hydrocarbons, aromatic compounds, amides, peroxides, azides, halides, oxyhalides, phosphides, phosphines, arsines, sulfides, thiols, boranes and/or hydrides. The reactive gas can be activated in the enclosure, e.g., by irradiation (e.g., electron beam, UV irradiation, microwave irradiation, heating, IR radiation), so that it reacts with the biomass. The biomass itself can be activated, for example by irradiation. Preferably the biomass is activated by the electron beam, to produce radicals which then react with the activated or unactivated reactive gas, e.g., by radical coupling or quenching.

Purging gases supplied to an enclosed conveyor can also be cooled, for example below about 25° C., below about 0° C., below about −40° C., below about −80° C., below about −120° C. For example, the gas can be boiled off from a compressed gas such as liquid nitrogen or sublimed from solid carbon dioxide. As an alternative example, the gas can be cooled by a chiller or part of or the entire conveyor can be cooled.

### Other Embodiments

Any material, processes or processed materials discussed herein can be used to make products and/or intermediates such as composites, fillers, binders, plastic additives, adsorbents and controlled release agents. The methods can include densification, for example, by applying pressure and heat to the materials. For example composites can be made by combining fibrous materials with a resin or polymer. For example radiation cross-linkable resin, e.g., a thermoplastic resin can be combined with a fibrous material to provide a fibrous material/cross-linkable resin combination. Such materials can be, for example, useful as building materials, protective sheets, containers and other structural materials (e.g., molded and/or extruded products). Absorbents can be, for example, in the form of pellets, chips, fibers and/or sheets. Adsorbents can be used, for example, as pet bedding, packaging material or in pollution control systems. Controlled release matrices can also be the form of, for example, pellets, chips, fibers and or sheets. The controlled release matrices can, for example, be used to release drugs, biocides, fragrances. For example, composites, absorbents and control release agents and their uses are described in U.S. Serial No. PCT/US2006/010648, filed Mar. 23, 2006, and U.S. Pat. No. 8,074,910 filed Nov. 22, 2011, the entire disclosures of which are herein incorporated by reference.

In some instances the biomass material is treated at a first level to reduce recalcitrance, e.g., utilizing accelerated electrons, to selectively release one or more sugars (e.g., xylose). The biomass can then be treated to a second level to release one or more other sugars (e.g., glucose). Optionally the biomass can be dried between treatments. The treatments can include applying chemical and biochemical treatments to release the sugars. For example, a biomass material can be treated to a level of less than about 20 Mrad (e.g., less than about 15 Mrad, less than about 10 Mrad, less than about 5 Mrad, less than about 2 Mrad) and then treated with a solution of sulfuric acid, containing less than 10% sulfuric acid (e.g., less than about 9%, less than about 8%, less than about 7%, less than about 6%, less than about 5%, less than about 4%, less than about 3%, less than about 2%, less than about 1%, less than about 0.75%, less than about 0.50%, less than about 0.25%) to release xylose. Xylose, for example that is released into solution, can be separated from solids and optionally the solids washed with a solvent/solution (e.g., with water and/or acidified water). Optionally, the solids can be dried, for example in air and/or under vacuum optionally with heating (e.g., below about 150 deg C., below about 120 deg C.) to a water content below about 25 wt % (below about 20 wt. %, below about 15 wt. %, below about 10 wt. %, below about 5 wt. %). The solids can then be treated with a level of less than about 30 Mrad (e.g., less than about 25 Mrad, less than about 20 Mrad, less than about 15 Mrad, less than about 10 Mrad, less than about 5 Mrad, less than about 1 Mrad or even not at all) and then treated with an enzyme (e.g., a cellulase) to release glucose. The glucose (e.g., glucose in solution) can be separated from the remaining solids. The solids can then be further processed, for example utilized to make energy or other products (e.g., lignin derived products).

### Flavors, Fragrances and Colorants

Any of the products and/or intermediates described herein, for example, produced by the processes, systems and/or equipment described herein, can be combined with flavors, fragrances, colorants and/or mixtures of these. For example, any one or more of (optionally along with flavors, fragrances and/or colorants) sugars, organic acids, fuels, polyols, such as sugar alcohols, biomass, fibers and composites can be combined with (e.g., formulated, mixed or reacted) or used to make other products. For example, one or more such product can be used to make soaps, detergents, candies, drinks (e.g., cola, wine, beer, liquors such as gin or vodka, sports drinks, coffees, teas), pharmaceuticals, adhesives, sheets (e.g., woven, none woven, filters, tissues) and/or composites (e.g., boards). For example, one or more such product can be combined with herbs, flowers, petals, spices, vitamins, potpourri, or candles. For example, the formulated, mixed or reacted combinations can have flavors/fragrances of grapefruit, orange, apple, raspberry, banana, lettuce, celery, cinnamon, chocolate, vanilla, peppermint, mint, onion, garlic, pepper, saffron, ginger, milk, wine, beer, tea, lean beef, fish, clams, olive oil, coconut fat, pork fat, butter fat, beef bouillon, legume, potatoes, marmalade, ham, coffee and cheeses.

Flavors, fragrances and colorants can be added in any amount, such as between about 0.001 wt. % to about 30 wt. %, e.g., between about 0.01 to about 20, between about 0.05 to about 10, or between about 0.1 wt. % to about 5 wt. %. These can be formulated, mixed and or reacted (e.g., with any one of more product or intermediate described herein) by any means and in any order or sequence (e.g., agitated, mixed, emulsified, gelled, infused, heated, sonicated, and/or suspended). Fillers, binders, emulsifier, antioxidants can also be utilized, for example protein gels, starches and silica.

In one embodiment the flavors, fragrances and colorants can be added to the biomass immediately after the biomass is irradiated such that the reactive sites created by the irradiation may react with reactive compatible sites of the flavors, fragrances, and colorants.

The flavors, fragrances and colorants can be natural and/or synthetic materials. These materials can be one or more of a compound, a composition or mixtures of these (e.g., a formulated or natural composition of several compounds). Optionally the flavors, fragrances, antioxidants and colorants can be derived biologically, for example, from a fermentation process (e.g., fermentation of saccharified materials as described herein). Alternatively, or additionally these flavors, fragrances and colorants can be harvested from a whole organism (e.g., plant, fungus, animal, bacteria or yeast) or a part of an organism. The organism can be collected and or extracted to provide color, flavors, fragrances and/or antioxidant by any means including utilizing the methods, systems and equipment described herein, hot water extraction, supercritical fluid extraction, chemical extraction (e.g., solvent or reactive extraction including acids and bases), mechanical extraction (e.g., pressing, comminuting, filtering), utilizing an enzyme, utilizing a bacteria such as to break down a starting material, and combinations of these methods. The compounds can be derived by a chemical reaction, for example, the combination of a sugar (e.g., as produced as described herein) with an amino acid (Maillard reaction). The flavor, fragrance, antioxidant and/or colorant can be an intermediate and or product produced by the methods, equipment or systems described herein, for example and ester and a lignin derived product.

Some examples of flavor, fragrances or colorants are polyphenols. Polyphenols are pigments responsible for the red, purple and blue colorants of many fruits, vegetables, cereal grains, and flowers. Polyphenols also can have antioxidant properties and often have a bitter taste. The antioxidant properties make these important preservatives. On class of polyphenols are the flavonoids, such as Anthocyanidines, flavanonols, flavan-3-ols, s, flavanones and flavanonols. Other phenolic compounds that can be used include phenolic acids and their esters, such as chlorogenic acid and polymeric tannins.

Among the colorants inorganic compounds, minerals or organic compounds can be used, for example titanium dioxide, zinc oxide, aluminum oxide, cadmium yellow (E.g., CdS), cadmium orange (e.g., CdS with some Se), alizarin crimson (e.g., synthetic or non-synthetic rose madder), ultramarine (e.g., synthetic ultramarine, natural ultramarine, synthetic ultramarine violet), cobalt blue, cobalt yellow, cobalt green, viridian (e.g., hydrated chromium(III)oxide), chalcophylite, conichalcite, cornubite, cornwallite and liroconite. Black pigments such as carbon black and self-dispersed blacks may be used.

Some flavors and fragrances that can be utilized include ACALEA TBHQ, ACET C-6, ALLYL AMYL GLYCOLATE, ALPHA TERPINEOL, AMBRETTOLIDE, AMBRINOL 95, ANDRANE, APHERMATE, APPLELIDE, BACDANOL®, BERGAMAL, BETA IONONE EPDXIDE, BETA NAPHTHYL ISO-BUTYL ETHER, BICYCLONONALACTONE, BORNAFIX®, CANTHOXAL, CASHMERAN®, CASHMERAN® VELVET, CASSIFFIX®, CEDRAFIX, CEDRAMBER®, CEDRYL ACETATE, CELESTOLIDE, CINNAMALVA, CITRAL DIMETHYL ACETATE, CITROLATE™, CITRONELLOL 700, CITRONELLOL 950, CITRONELLOL COEUR, CITRONELLYL ACETATE, CITRONELLYL ACETATE PURE, CITRONELLYL FORMATE, CLARYCET, CLONAL, CONIFERAN, CONIFERAN PURE, CORTEX ALDEHYDE 50% PEOMOSA, CYCLABUTE, CYCLACET®, CYCLAPROP®, CYCLEMAX™, CYCLOHEXYL ETHYL ACETATE, DAMASCOL, DELTA DAMASCONE, DIHYDRO CYCLACET, DIHYDRO MYRCENOL, DIHYDRO TERPINEOL, DIHYDRO TERPINYL ACETATE, DIMETHYL CYCLORMOL, DIMETHYL OCTANOL PQ, DIMYRCETOL, DIOLA, DIPENTENE, DULCINYL® RECRYSTALLIZED, ETHYL-3-PHENYL GLYCIDATE, FLEURAMONE, FLEURANIL, FLORAL SUPER, FLORALOZONE, FLORIFFOL, FRAISTONE, FRUCTONE, GALAXOLIDE® 50, GALAXOLIDE® 50 BB, GALAXOLIDE® 50 IPM, GALAXOLIDE® UNDILUTED, GALBASCONE, GERALDEHYDE, GERANIOL 5020, GERANIOL 600 TYPE, GERANIOL 950, GERANIOL 980 (PURE), GERANIOL CFT COEUR, GERANIOL COEUR, GERANYL ACETATE COEUR, GERANYL ACETATE, PURE, GERANYL FORMATE, GRISALVA, GUAIYL ACETATE, HELIONAL™ HERBAC, HERBALIME™, HEXADECANOLIDE, HEXALON, HEXENYL SALICYLATE CIS 3-, HYACINTH BODY, HYACINTH BODY NO. 3, HYDRATROPIC ALDEHYDE.DMA, HYDROXYOL, INDOLAROME, INTRELEVEN ALDEHYDE, INTRELEVEN ALDEHYDE SPECIAL, IONONE ALPHA, IONONE BETA, ISO CYCLO CITRAL, ISO CYCLO GERANIOL, ISO E SUPER®, ISOBUTYL QUINOLINE, JASMAL, JESSEMAL®, KHARISMAL®, KHARISMAL® SUPER, KHUSINIL, KOAVONE®, KOHINOOL®, LIFFAROME™, LIMOXAL, LINDENOL™, LYRAL®, LYRAME SUPER, MANDARIN ALD 10% TRI ETH, CITR, MARITIMA, MCK CHINESE, MEIJIFF™, MELAFLEUR, MELOZONE, METHYL ANTHRANILATE, METHYL IONONE ALPHA EXTRA, METHYL IONONE GAMMA A, METHYL IONONE GAMMA COEUR, METHYL IONONE GAMMA PURE, METHYL LAVENDER KETONE, MONTAVERDI®, MUGUESIA, MUGUET ALDEHYDE 50, MUSK Z4, MYRAC ALDEHYDE, MYRCENYL ACETATE, NECTARATE™, NEROL 900, NERYL ACETATE, OCIMENE, OCTACETAL, ORANGE FLOWER ETHER, ORIVONE, ORRINIFF 25%, OXASPIRANE, OZOFLEUR, PAMPLEFLEUR®, PEOMOSA, PHENOXANOL®, PICONIA, PRECYCLEMONE B, PRENYL ACETATE, PRISMANTOL, RESEDA BODY, ROSALVA, ROSAMUSK, SANJINOL, SANTALIFF™, SYVERTAL, TERPINEOL,TERPINOLENE 20, TERPINOLENE 90 PQ, TERPINOLENE RECT., TERPINYL ACETATE, TERPINYL ACETATE JAX, TETRAHYDRO, MUGUOL®, TETRAHYDRO MYRCENOL, TETRAMERAN, TIMBERSILK™, TOBACAROL, TRIMOFIX® 0 TT, TRIPLAL®, TRISAMBER®, VANORIS, VERDOX™, VERDOX™ HC, VERTENEX®, VERTENEX® HC, VERTOFIX® COEUR, VERTOLIFF, VERTOLIFF ISO, VIOLIFF, VIVALDIE, ZENOLIDE, ABS INDIA 75 PCT MIGLYOL, ABS MOROCCO 50 PCT DPG, ABS MOROCCO 50 PCT TEC, ABSOLUTE FRENCH, ABSOLUTE INDIA, ABSOLUTE MD 50 PCT BB, ABSOLUTE MOROCCO, CONCENTRATE PG, TINCTURE 20 PCT, AMBERGRIS, AMBRETTE ABSOLUTE, AMBRETTE SEED OIL, ARMOISE OIL 70 PCT THUYONE, BASIL ABSOLUTE GRAND VERT, BASIL GRAND VERT ABS MD, BASIL OIL GRAND VERT, BASIL OIL VERVEINA, BASIL OIL VIETNAM, BAY OIL TERPENELESS, BEESWAX ABS N G, BEESWAX ABSOLUTE, BENZOIN RESINOID SIAM, BENZOIN RESINOID SIAM 50 PCT DPG, BENZOIN RESINOID SIAM 50 PCT PG, BENZOIN RESINOID SIAM 70.5 PCT TEC, BLACKCURRANT BUD ABS 65 PCT PG, BLACKCURRANT BUD ABS MD 37 PCT TEC, BLACKCURRANT BUD ABS MIGLYOL, BLACKCURRANT BUD ABSOLUTE BURGUNDY, BOIS DE ROSE OIL, BRAN ABSOLUTE, BRAN RESINOID, BROOM ABSOLUTE ITALY, CARDAMOM GUATEMALA CO2 EXTRACT, CARDAMOM OIL GUATEMALA, CARDAMOM OIL INDIA, CARROT HEART, CASSIE ABSOLUTE EGYPT, CASSIE ABSOLUTE MD 50 PCT IPM, CASTOREUM ABS 90 PCT TEC, CASTOREUM ABS C 50 PCT MIGLYOL, CASTOREUM ABSOLUTE, CASTOREUM RESINOID, CASTOREUM RESINOID 50 PCT DPG, CEDROL CEDRENE, CEDRUS ATLANTICA OIL REDIST, CHAMOMILE OIL ROMAN, CHAMOMILE OIL WILD, CHAMOMILE OIL WILD LOW LIMONENE, CINNAMON BARK OIL CEYLAN, CISTE ABSOLUTE, CISTE ABSOLUTE COLORLESS, CITRONELLA OIL ASIA IRON FREE, CIVET ABS 75 PCT PG, CIVET ABSOLUTE, CIVET TINCTURE 10 PCT, CLARY SAGE ABS FRENCH DECOL, CLARY SAGE ABSOLUTE FRENCH, CLARY SAGE C′LESS 50 PCT PG, CLARY SAGE OIL FRENCH, COPAIBA BALSAM, COPAIBA BALSAM OIL, CORIANDER SEED OIL, CYPRESS OIL, CYPRESS OIL ORGANIC, DAVANA OIL, GALBANOL, GALBANUM ABSOLUTE COLORLESS, GALBANUM OIL, GALBANUM RESINOID, GALBANUM RESINOID 50 PCT DPG, GALBANUM RESINOID HERCOLYN BHT, GALBANUM RESINOID TEC BHT, GENTIANE ABSOLUTE MD 20 PCT BB, GENTIANE CONCRETE, GERANIUM ABS EGYPT MD, GERANIUM ABSOLUTE EGYPT, GERANIUM OIL CHINA, GERANIUM OIL EGYPT, GINGER OIL 624, GINGER OIL RECTIFIED SOLUBLE, GUAIACWOOD HEART, HAY ABS MD 50 PCT BB, HAY ABSOLUTE, HAY ABSOLUTE MD 50 PCT TEC, HEALINGWOOD, HYSSOP OIL ORGANIC, IMMORTELLE ABS YUGO MD 50 PCT TEC, IMMORTELLE ABSOLUTE SPAIN, IMMORTELLE ABSOLUTE YUGO, JASMIN ABS INDIA MD, JASMIN ABSOLUTE EGYPT, JASMIN ABSOLUTE INDIA, ASMIN ABSOLUTE MOROCCO, JASMIN ABSOLUTE SAMBAC, JONQUILLE ABS MD 20 PCT BB, JONQUILLE ABSOLUTE France, JUNIPER BERRY OIL FLG, JUNIPER BERRY OIL RECTIFIED SOLUBLE, LABDANUM RESINOID 50 PCT TEC, LABDANUM RESINOID BB, LABDANUM RESINOID MD, LABDANUM RESINOID MD 50 PCT BB, LAVANDIN ABSOLUTE H, LAVANDIN ABSOLUTE MD, LAVANDIN OIL ABRIAL ORGANIC, LAVANDIN OIL GROSSO ORGANIC, LAVANDIN OIL SUPER, LAVENDER ABSOLUTE H, LAVENDER ABSOLUTE MD, LAVENDER OIL COUMARIN FREE, LAVENDER OIL COUMARIN FREE ORGANIC, LAVENDER OIL MAILLETTE ORGANIC, LAVENDER OIL MT, MACE ABSOLUTE BB, MAGNOLIA FLOWER OIL LOW METHYL EUGENOL, MAGNOLIA FLOWER OIL, MAGNOLIA FLOWER OIL MD, MAGNOLIA LEAF OIL, MANDARIN OIL MD, MANDARIN OIL MD BHT, MATE ABSOLUTE BB, MOSS TREE ABSOLUTE MD TEX IFRA 43, MOSS-OAK ABS MD TEC IFRA 43, MOSS-OAK ABSOLUTE IFRA 43, MOSS-TREE ABSOLUTE MD IPM IFRA 43, MYRRH RESINOID BB, MYRRH RESINOID MD, MYRRH RESINOID TEC, MYRTLE OIL IRON FREE, MYRTLE OIL TUNISIA RECTIFIED, NARCISSE ABS MD 20 PCT BB, NARCISSE ABSOLUTE FRENCH, NEROLI OIL TUNISIA, NUTMEG OIL TERPENELESS, OEILLET ABSOLUTE, OLIBANUM RESINOID, OLIBANUM RESINOID BB, OLIBANUM RESINOID DPG, OLIBANUM RESINOID EXTRA 50 PCT DPG, OLIBANUM RESINOID MD, OLIBANUM RESINOID MD 50 PCT DPG, OLIBANUM RESINOID TEC, OPOPONAX RESINOID TEC, ORANGE BIGARADE OIL MD BHT, ORANGE BIGARADE OIL MD SCFC, ORANGE FLOWER ABSOLUTE TUNISIA, ORANGE FLOWER WATER ABSOLUTE TUNISIA, ORANGE LEAF ABSOLUTE, ORANGE LEAF WATER ABSOLUTE TUNISIA, ORRIS ABSOLUTE ITALY, ORRIS CONCRETE 15 PCT IRONE, ORRIS CONCRETE 8 PCT IRONE, ORRIS NATURAL 15 PCT IRONE 4095C, ORRIS NATURAL 8 PCT IRONE 2942C, ORRIS RESINOID, OSMANTHUS ABSOLUTE, OSMANTHUS ABSOLUTE MD 50 PCT BB, PATCHOULI HEART No 3, PATCHOULI OIL INDONESIA, PATCHOULI OIL INDONESIA IRON FREE, PATCHOULI OIL INDONESIA MD, PATCHOULI OIL REDIST, PENNYROYAL HEART, PEPPERMINT ABSOLUTE MD, PETITGRAIN BIGARADE OIL TUNISIA, PETITGRAIN CITRONNIER OIL, PETITGRAIN OIL PARAGUAY TERPENELESS, PETITGRAIN OIL TERPENELESS STAB, PIMENTO BERRY OIL, PIMENTO LEAF OIL, RHODINOL EX GERANIUM CHINA, ROSE ABS BULGARIAN LOW METHYL EUGENOL, ROSE ABS MOROCCO LOW METHYL EUGENOL, ROSE ABS TURKISH LOW METHYL EUGENOL, ROSE ABSOLUTE, ROSE ABSOLUTE BULGARIAN, ROSE ABSOLUTE DAMASCENA, ROSE ABSOLUTE MD, ROSE ABSOLUTE MOROCCO, ROSE ABSOLUTE TURKISH, ROSE OIL BULGARIAN, ROSE OIL DAMASCENA LOW METHYL EUGENOL, ROSE OIL TURKISH, ROSEMARY OIL CAMPHOR ORGANIC, ROSEMARY OIL TUNISIA, SANDALWOOD OIL INDIA, SANDALWOOD OIL INDIA RECTIFIED, SANTALOL, SCHINUS MOLLE OIL, ST JOHN BREAD TINCTURE 10 PCT, STYRAX RESINOID, STYRAX RESINOID, TAGETE OIL, TEA TREE HEART, TONKA BEAN ABS 50 PCT SOLVENTS, TONKA BEAN ABSOLUTE, TUBEROSE ABSOLUTE INDIA, VETIVER HEART EXTRA, VETIVER OIL HAITI, VETIVER OIL HAITI MD, VETIVER OIL JAVA, VETIVER OIL JAVA MD, VIOLET LEAF ABSOLUTE EGYPT, VIOLET LEAF ABSOLUTE EGYPT DECOL, VIOLET LEAF ABSOLUTE FRENCH, VIOLET LEAF ABSOLUTE MD 50 PCT BB, WORMWOOD OIL TERPENELESS, YLANG EXTRA OIL, YLANG III OIL and combinations of these.

The colorants can be among those listed in the Color Index International by the Society of Dyers and Colourists. Colorants include dyes and pigments and include those commonly used for coloring textiles, paints, inks and inkjet inks. Some colorants that can be utilized include carotenoids, arylide yellows, diarylide yellows, ß-naphthols, naphthols, benzimidazolones, disazo condensation pigments, pyrazolones, nickel azo yellow, phthalocyanines, quinacridones, perylenes and perinones, isoindolinone and isoindoline pigments, triarylcarbonium pigments, diketopyrrolo-pyrrole pigments, thioindigoids. Cartenoids include, for example, alpha-carotene, beta-carotene, gamma-carotene, lycopene, lutein and astaxanthin, Annatto extract, Dehydrated beets (beet powder), Canthaxanthin, Caramel, β-Apo-8′-carotenal, Cochineal extract, Carmine, Sodium copper chlorophyllin, Toasted partially defatted cooked cottonseed flour, Ferrous gluconate, Ferrous lactate, Grape color extract, Grape skin extract (enocianina), Carrot oil, Paprika, Paprika oleoresin, Mica-based pearlescent pigments, Riboflavin, Saffron, Titanium dioxide, Tomato lycopene extract; tomato lycopene concentrate, Turmeric, Turmeric oleoresin, FD&C Blue No. 1, FD&C Blue No. 2, FD&C Green No. 3, Orange B, Citrus Red No. 2, FD&C Red No. 3, FD&C Red No. 40, FD&C Yellow No. 5, FD&C Yellow No. 6, Alumina (dried aluminum hydroxide), Calcium carbonate, Potassium sodium copper chlorophyllin (chlorophyllin-copper complex), Dihydroxyacetone, Bismuth oxychloride, Ferric ammonium ferrocyanide, Ferric ferrocyanide, Chromium hydroxide green, Chromium oxide greens, Guanine, Pyrophyllite, Talc, Aluminum powder, Bronze powder, Copper powder, Zinc oxide, D&C Blue No. 4, D&C Green No. 5, D&C Green No. 6, D&C Green No. 8, D&C Orange No. 4, D&C Orange No. 5, D&C Orange No. 10, D&C Orange No. 11, FD&C Red No. 4, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Red No. 39, D&C Violet No. 2, D&C Yellow No. 7, Ext. D&C Yellow No. 7, D&C Yellow No. 8, D&C Yellow No. 10, D&C Yellow No. 11, D&C Black No. 2, D&C Black No. 3 (3), D&C Brown No. 1, Ext. D&C, Chromium-cobalt-aluminum oxide, Ferric ammonium citrate, Pyrogallol, Logwood extract, 1,4-Bis[(2-hydroxy-ethyeamino]-9,10-anthracenedione bis(2-propenoic)ester copolymers, 1,4-Bis [(2-methylphenyl)amino]-9,10-anthracenedione, 1,4-Bis[4-(2-methacryloxyethyl) phenylamine] anthraquinone copolymers, Carbazole violet, Chlorophyllin-copper complex, Chromium-cobalt-aluminum oxide, C.I. Vat Orange 1, 2-[[2,5-Diethoxy-4-[(4-methylphenyl)thiol] phenyl]azo]-1,3,5-benzenetriol, 16,23-Dihydrodinaphtho [2,3-a:2′,3′-i] naphth [2′,3′: 6,7] indolo [2,3-c] carbazole-5,10,15,17,22,24-hexone, N,N′-(9,10-Dihydro-9,10-dioxo-1,5-anthracenediyl) bisbenzamide, 7,16-Dichloro-6,15-dihydro-5,9,14,18-anthrazinetetrone, 16,17-Dimethoxydinaphtho (1,2,3-cd:3′,2′,1′-lm) perylene-5,10-dione, Poly(hydroxyethyl methacrylate)-dye copolymers(3), Reactive Black 5, Reactive Blue 21, Reactive Orange 78, Reactive Yellow 15, Reactive Blue No. 19, Reactive Blue No. 4, C.I. Reactive Red 11, C.I. Reactive Yellow 86, C.I. Reactive Blue 163, C.I. Reactive Red 180, 4-[(2,4-dimethylphenyl)azo]-2,4-dihydro-5-methyl-2-phenyl-3H-pyrazol-3-one (solvent Yellow 18), 6-Ethoxy-2-(6-ethoxy-3-oxobenzo[b] thien-2(3H)-ylidene) benzo[b]thiophen-3(2H)-one, Phthalocyanine green, Vinyl alcohol/methyl methacrylate-dye reaction products, C.I. Reactive Red 180, C.I. Reactive Black 5, C.I. Reactive Orange 78, C.I. Reactive Yellow 15, C.I. Reactive Blue 21, Disodium 1-amino-4-[[4-[(2-bromo-1-oxoallyl)amino]-2-sulphonatophenyl]amino]-9,10-dihydro-9,10-dioxoanthracene-2-sulphonate (Reactive Blue 69), D&C Blue No. 9, [Phthalocyaninato(2-)] copper and mixtures of these.

Other than in the examples herein, or unless otherwise expressly specified, all of the numerical ranges, amounts, values and percentages, such as those for amounts of materials, elemental contents, times and temperatures of reaction, ratios of amounts, and others, in the following portion of the specification and attached claims may be read as if prefaced by the word “about” even though the term “about” may not expressly appear with the value, amount, or range. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains error necessarily resulting from the standard deviation found in its underlying respective testing measurements. Furthermore, when numerical ranges are set forth herein, these ranges are inclusive of the recited range end points (e.g., end points may be used). When percentages by weight are used herein, the numerical values reported are relative to the total weight.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10. The terms “one,” “a,” or “an” as used herein are intended to include “at least one” or “one or more,” unless otherwise indicated.

Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

While this invention has been particularly shown and described with references to preferred embodiments thereof, it will be understood by those skilled in the art that various changes in form and details may be made therein without departing from the scope of the invention encompassed by the appended claims.

1.-20. (canceled)

21. A method to purify a bioprocessed feedstock, wherein the bioprocessed feedstock has been produced by a method comprising saccharification of biomass material to sugars and fermentation of the sugars, the method comprising:
producing a first aqueous permeate from a bioprocessed feedstock containing (a) yeast and/or bacterial cells, and (b) oligomers, large molecules and/or inorganic clusters, by retaining material from the bioprocessed feedstock with a molecular weight above about 100 kDa utilizing a first membrane filter, and
producing a second aqueous permeate from the first permeate by retaining from the first permeate material with a molecular weight above about 5 kDa utilizing a second membrane filter.

22. The method of claim 1, wherein saccharification is done by contacting the biomass material with an enzyme or organism.

23. The method of claim 1, wherein the biomass material is a cellulosic or lignocellulosic material.

24. The method of claim 1, wherein the bioprocessed feedstock is in the form of a slurry that includes less than about 1% solids.

25. The method of claim 24, wherein the solids have a median particle size of less than about 10 μm.

26. The method of claim 1 wherein the bioprocessed feedstock has been filtered prior to producing the first aqueous permeate.

27. The method of claim 26, wherein the bioprocessed feedstock includes at least about 1% solids before being filtered.

28. The method of claim 26, wherein the bioprocessed feedstock has been filtered by a method selected from decanter centrifuging, disc centrifuging, stack filtration, plate filtration, micro filtration, column filtration, Vibratory Enhanced Separation Processes and combinations of these.

29. The method of claim 1, wherein the bioprocessed feedstock comprises enzymes, cellular debris, partial hydrolysis products of cellulose and hemicellulose, inorganic solids and colored impurities.

30. The method of claim 1, wherein the bioprocessed feedstock has had at least one volatile compound removed therefrom under vacuum prior to the bioprocessed feedstock being filtered by the first membrane filter.

31. The method of claim 30, wherein the volatile compound is an ester.

32. The method of claim 30, wherein the volatile compound is an alcohol.

33. The method of claim 30, wherein the concentration of alcohol in the bioprocessed feedstock is less than about 5%.

34. The method of claim 1, wherein the first membrane filter retains particles larger than about 0.05 μm.

35. The method of claim 1, wherein the second membrane filter retains solids above about 5 nanometers.

36. The method of claim 1, wherein the first and second membrane filters are configured as cross flow filters.

37. The method of claim 36, wherein the first and/or second membrane filters are spiral wound filters, tube filters or hollow fiber filters.

38. The method of claim 36, wherein the first and/or second membrane filters are configured as tube filters with a diameter between about ¼ and about 1 inch.

39. The method of claim 36, wherein the inlet pressure at the first and/or the second membrane filter is between about 90 and about 500 PSIG and the outlet pressure at the first and/or second membrane filter is between about 20 and about 430 PSIG.

40. The method of claim 38, wherein the bioprocessed feedstock flows through the tube filter(s) at a flow rate between about 1 and about 20 gallons per minute.
